(19) United States
(12) Patent Application Publication
(10) Pub. No.: US 2026/0229363 A1
(43) Pub. Date: Aug. 6, 2026

(54) METHOD AND SYSTEM FOR OBTAINING ADVERSE OUTCOME INFORMATION

(71) Applicants: University of Strathclyde, Glasgow (GB); KING'S COLLEGE LONDON, London (GB)

(72) Inventors: Tunde Csoban, Glasgow (GB); Paul Murray, Glasgow (GB); Kimberley Kavanagh, Glasgow (GB); Laura A. Magee, London (GB); Peter von Dadelszen, London (GB)

(73) Assignees: University of Strathclyde, Glasgow, JJ (GB); KING'S COLLEGE LONDON, LONDON (GB)

(21) Appl. No.: 19/148,532

(22) PCT Filed: Jan. 18, 2024
(86) PCT No.: PCT/GB2024/050141

§ 371 Date: Jul. 16, 2025

(30) Foreign Application Priority Data
Jan. 19, 2023 (GB) 2300798.2

(51) Int. Cl. G16H 50/30; G06N 20/00; G16H 50/70

(52) U.S. Cl. CPC G16H 50/30; G06N 20/00; G16H 50/70

(57) ABSTRACT

A method of obtaining a risk level and/or a probability associated with one or more adverse maternal outcomes for a subject with suspected or confirmed pre-eclampsia comprising: obtaining input data comprising at least subject data for the subject, wherein the subject data comprises a combination of at least two of: demographic data; vital sign data representative of one or more vital signs of the subject; and sample data representative of one or more parameters obtainable from an analysis of a sample; providing the obtained input data to a machine learning derived procedure configured to obtain the risk level and/or probability associated with one or more adverse maternal outcomes for the subject, wherein the input data further comprises health care system data representing a value for at least one statistic or indicator associated with the health care system and/or a country and/or a region of the health care system.

### FIELD

The present invention relates to a method and system for obtaining adverse maternal outcome information, for example, a risk level and/or probability associated with one or more adverse maternal outcomes for a subject with suspected or confirmed preeclampsia.

### BACKGROUND

Pre-eclampsia is a pregnancy condition affecting 2 to 8% of pregnancies which can cause adverse maternal outcomes including 50,000-100,000 maternal deaths annually and serious morbidities. Current models recommended in clinical practice for prediction of adverse outcomes use logistic regression or survival analysis and are healthcare system specific.

An example of current models include the logistic regression-based Pre-eclampsia Integrated Estimate of RiSk (PIERS) model described in “Prediction of adverse maternal outcomes in pre-eclampsia: development and validation of the fullPIERS model” by von Dadelszen P et al., Lancet 2011; 377(9761): 219-27. The fullPIERS model was developed for well-resourced settings and using variables such as gestational age, symptoms (including chest pain or dyspnea), signs (oxygen saturation), and laboratory tests (platelet count, and creatinine and aspartate transaminase concentrations).

“A machine-learning-based algorithm improves prediction of preeclampsia-associated adverse outcomes” Am J Obstet Gynecol 2022; 227(1): 77 e1-e30, by Schmidt et al. describes an algorithm for determining a composite foetal and maternal adverse outcome at any time after a visit, up until 14 days after delivery of the neonate.

### SUMMARY

According to a first aspect, there is provided a method of obtaining a risk level and/or a probability associated with one or more adverse maternal outcomes for a subject with suspected or confirmed pre-eclampsia comprising: obtaining input data comprising at least subject data for the subject, wherein the subject data comprises a combination of at least two of: demographic data; vital sign data representative of one or more vital signs of the subject; and sample data representative of one or more parameters obtainable from an analysis of a sample; providing the obtained input data to a machine learning derived configured to obtain the risk level and/or probability associated with one or more adverse maternal outcomes for the subject, wherein the input data further comprises health care system data representing a value for at least one statistic or indicator associated with the health care system and/or a country and/or a region of the health care system.

The obtained input data may represent values for a reduced set of input variables, wherein the selected set of input variables are selected from a larger set of input variables in accordance with a feature reduction process. The input data may further comprise input representing an elapsed time and wherein the risk level and/or probability is obtained in dependence on at least the elapsed time.

The health care system data may comprise at least one non-clinical statistic or indicator. The health system data may comprise national and/or regional per capita gross domestic product and/or a national or regional maternal mortality ratio.

The obtained input data may represent values for a pre-determined set of input variables and the machine learning derive procedure may be trained to receive and process said input data.

The input data may be representative of values for a set of selected set of input features, wherein:
a) the health care system data are representative of: national per capita gross domestic product and/or national or regional maternal mortality ratio;
b) the demographic data are representative of: maternal age at expected date of delivery, gestational age at eligibility;
c) the vital sign data are representative of: height, weight at time of assessment, systolic blood pressure, diastolic blood pressure, oxygen saturation;
d) the sample data are representative of: haematology sample data comprising haematocrit, platelet count, total leukocyte count; renal sample data comprising: serum creatinine, uric acid; hepatic sample data comprising: lactate dehydrogenase, aspartate transaminase, alanine transaminase, serum albumin.

The obtained input data may be representative of values for a set of input variables such that the input data comprises:
vital sign data representative of values for at least some, optionally all, of the following input variables: oxygen saturation;
the sample data representative of values for at least some, optionally all, of the following input variables: platelet count haematocrit, serum albumin, serum creatinine, uric acid, aspartate transaminase, and alanine transaminase;
wherein the input data further comprises symptom data representative of values for at least some, optionally all, of the following input variables: a symptom of vomiting or nausea, a symptom of right upper quadrant or epigastric pain, a symptom of chest pain or dyspnoea.

The elapsed time may comprise a time elapsed from a pre-defined clinical event, for example, admission of the subject to a health care setting and/or an occurrence of a pre-determined symptom.

Obtaining the input data may comprise obtaining sample data for a first time and providing the sample data as part of first input data to the model to obtain the risk level and/or probability associated with the first time and obtaining sample data for a second, subsequent time and providing the sample data as part of the input data to the model to obtain the risk level and/or probability associated with the second time

The machine learning derived procedure may comprise at least one part configured to take into account a magnitude and rate of change of the obtained input data.

The machine learning derived procedure may comprise a combination of one or more of random forest and/or regression models with a generalised linear mixed model, optionally, wherein the machine learning derived procedure comprises a combination of a Bayesian generalized linear mixed model (GLMM) and a random forest model.

The input data may not include symptom data representative of one or more symptoms of the subject.

The input data may comprise data representing values for at least the following input variables: national per capita gross domestic product, maternal mortality ratio, systolic and diastolic blood pressure, uric acid.

The health system data may be representative of at least one of the following input variables: national per capita gross domestic product, national maternal mortality ratio. The demographic data may be representative of at least one of the following input variables: maternal age at expected date of delivery, gestational age at eligibility. The vital sign data may be representative of at least one of the following input variables: height, weight at time of assessment, systolic blood pressure, diastolic blood pressure, oxygen saturation. The sample data may be representative of at least one of the following input variables: hematology sample data comprising hematocrit, platelet count, total leukocyte count; renal sample data comprises: serum creatinine, uric acid; hepatic sample data comprising: lactate dehydrogenase, aspartate transaminase, alanine transaminase, serum albumin.

The one or more feature reduction processes may comprise a recursive feature elimination process based on importance scores for the larger set of parameters.

The machine learning derived procedure may comprise a random-forest procedure.

Obtaining input data may comprise at least one of:
a) receiving user input data representative of the country and/or region of the health care system and retrieving the health care system data representing a value for at least one statistic associated with the country and/or region of the health care system;
b) performing a vital sign measurement to obtain the vital sign data;
c) receiving user input data representative of the demographic data.

Obtaining the input data may comprise obtaining a sample from the subject and performing a sample analysis on the sample to obtain at least some of the sample data.

The sample may comprise a blood sample and wherein the sample analysis comprises a blood sample analysis to obtain at least one of: a) one or more haematological parameters including haematocrit, platelet count, total leukocyte count; b) one or more renal parameters including lactate dehydrogenase, aspartate transaminase, alanine transaminase, serum albumin.

The method may further comprise receiving user input data representing at least some input data and/or displaying the obtained risk level and/or probability. User input data data may be received via one or more user input devices.

The risk level and/or probability may represent the risk level and/or probability of an occurrence of the adverse maternal outcome within one or more predefined time periods.

The predefined time period may comprise two days and/or seven days.

The adverse maternal outcome may comprise at least one of: maternal death; an adverse central nervous system event; a cardiorespiratory event; a hematologic event; a hepatic event; a renal event or one or more of: placental abruption, severe ascites, bell's palsy.

The machine learning derived procedure may be configured to classify the subject into one of a plurality of risk levels based on a probability of the occurrence of one or more adverse maternal events in a predetermined time period.

According to a second aspect, which may be provided independently, there is provided a method of obtaining a risk level and/or a probability associated with one or more adverse maternal outcomes for a subject with suspected or confirmed pre-eclampsia comprising: obtaining input data comprising at least subject data for the subject, wherein the subject data comprises a combination of at least two of: demographic data; vital sign data representative of one or more vital signs of the subject; and sample data representative of one or more parameters obtainable from an analysis of a sample; and providing the obtained input data to a machine learning derived configured to obtain the risk level and/or probability associated with one or more adverse maternal outcomes for the subject.

The obtained input data may represent values for a reduced set of input variables. The reduced set of input variables may be a reduced set of variables selected from a larger set of input variables in accordance with a feature reduction process.

The reduced set of features may comprise, optionally consist: a symptom of vomiting or nausea, a symptom of right upper quadrant or epigastric pain, a symptom of chest pain or dyspnoea, oxygen saturation, platelet count, haematocrit, serum albumin, serum creatinine, uric acid, aspartate transaminase, and alanine transaminase.

The obtained input data may be representative of values for a set of input variables such that the input data comprises:
vital sign data representative of values for at least some, optionally all, of the following input variables: oxygen saturation;
the sample data representative of values for at least some, optionally all, of the following input variables: platelet count, haematocrit, serum albumin, serum creatinine, uric acid, aspartate transaminase, and alanine transaminase;
wherein the input data further comprises symptom data representative of values for at least some, optionally all, of the following input variables: a symptom of vomiting or nausea, a symptom of right upper quadrant or epigastric pain, a symptom of chest pain or dyspnoea.

The input data may further comprise input representing an elapsed time and wherein the risk level and/or probability is obtained in dependence on at least the elapsed time.

According to a third aspect, which may be provided independently, there is provided a method of training a machine learning derived procedure comprising: obtaining training data associated with a plurality of subjects, wherein the training data comprises subject data, the subject data comprising a combination of at least two of: health system data; demographic data; symptom data representative of the presence of one or more symptoms; vital sign data representative of one or more vital signs of the subject and sample data representative of one or more parameters obtainable using an analysis of a sample; performing a training process using at least some of the training data to obtain a trained machine learning derived procedure configured to receive further input data comprising at least subject data for a further subject with suspected or confirmed preeclampsia and obtain a risk level and/or a probability associated with one or more adverse maternal outcomes for the subject, wherein the training data and further input data further comprises health care system data representing a value for at least one statistic associated with the health care system and/or a country and/or a region of the health care system.

The training process may comprise performing a feature reduction process to select a reduced set of input variables from a larger set of input variables and training the machine learning derived procedure to receive input data representative of values for the reduced set of input variables. The training data may be representative of data obtained over a time period, such that the trained machine learning derive procedure is configured to receive input representative of an elapsed time and a risk level and/or probability is obtained in dependence on at least the elapsed time.

The feature selection process may comprise: training one or more models using first data representative of a first group of features; determining importance scores for each feature of the first group of features; selecting the reduced set of features based on the determined importance scores.

The training method may comprise performing an iterative process comprising:
a) fitting a plurality of models of a first type using training data for a plurality of subjects over a plurality of time points using a target outcome;
b) fitting at least one model of a second type using at least predictions obtained from the plurality of models of the first type models and the target outcome;
c) using an output from the second model to update the target outcome for fitting the plurality of models of the first type.

The first type may comprise a random forest based model and/or the second type may comprise a generalized linear mixed model.

The method may further comprise:
a) fitting a random forest based model for each subject at each time point of a plurality of time points based on a target outcome;
b) obtaining a first predicted probability for each subject and time point from the random forest model;
c) fitting a Bayesian generalized linear mixed model (GLMM) using the probabilities obtained from the random forest models and the target outcome;
d) obtaining a second predicted probability for each subject and time point from the GLMM using the input data;
e) updating a target outcome using the second predicted probability, wherein updating the target outcome comprises adding the new prediction to the previous binary outcome and applying a split function to convert to a binary value;
f) repeating steps a) to e) until a convergence condition is satisfied.

The training data may comprise at least one of: training data for subjects with pre-eclampsia prior to 34 weeks gestation; training data for subjects in sub-Saharan Africa, North and South America, South Asia, Europe and Oceania.

The method may further comprise identifying missing data and replacing missing data using a missing data replacement scheme.

According to a fourth aspect, which may be provided independently, there is provided a method of training a machine learning derived procedure comprising: obtaining training data associated with a plurality of subjects, wherein the training data comprises subject data, the subject data comprising a combination of at least two of: health system data; demographic data; symptom data representative of the presence of one or more symptoms; vital sign data representative of one or more vital signs of the subject and sample data representative of one or more parameters obtainable using an analysis of a sample; performing a training process using at least some of the training data to obtain a trained machine learning derived procedure configured to receive input data comprising further subject data for a further subject with preeclampsia and obtain a risk level and/or a probability associated with one or more adverse maternal outcomes for the subject.

The method may comprise performing a feature reduction process to select a reduced set of input variables from a larger set of input variables and training the machine learning derived procedure to receive subject data representative of values for the reduced set of input variables.

The reduced set of features may comprise: a symptom of vomiting or nausea, a symptom of right upper quadrant or epigastric pain, a symptom of chest pain or dyspnoea, oxygen saturation, platelet count, haematocrit, serum albumin, serum creatinine, uric acid, aspartate transaminase, and alanine transaminase.

The input data may be representative of values for a set of input variables such that the input data comprises:
vital sign data representative of values for at least some, optionally all, of the following input variables: oxygen saturation;
the sample data representative of values for at least some, optionally all, of the following input variables: platelet count, haematocrit, serum albumin, serum creatinine, uric acid, aspartate transaminase, and alanine transaminase;
wherein the input data further comprises symptom data representative of values for at least some, optionally all, of the following input variables: a symptom of vomiting or nausea, a symptom of right upper quadrant or epigastric pain, a symptom of chest pain or dyspnoea.

The training data may be representative of data obtained over a time period, such that the trained machine learning derive procedure is configured to receive input representative of an elapsed time and the risk level and/or probability is obtained in dependence on at least the elapsed time.

According to a fifth aspect, that may be provided independently, there is provided an apparatus comprising a processing resource configured to perform the method of the any of the first to fourth aspects.

The apparatus may further comprise a user input device configured to receive user input data representing values for input variables. The apparatus may comprise a display for displaying the risk level and/or probability.

According to a sixth aspect, there is provided a computer program product comprising computer-readable instructions that are executable to perform the method of any of the first to fourth aspects.

Features in one aspect may be applied as features in any other aspect, in any appropriate combination. For example, features of the first aspect may be provided as features of the second to the sixth aspect and vice versa. For example, method features may be provided as apparatus features and/or computer program product features or vice versa.

### BRIEF DESCRIPTION OF DRAWINGS

Various aspects of the invention will now be described by way of example only, and with reference to the accompanying drawings, of which:

FIG. 1 is a schematic diagram of a data processing system in accordance with an embodiment;

FIG. 2 is a schematic diagram showing in overview a trained machine learning procedure for obtaining a risk level and/or a probability associated with one or more adverse maternal outcomes for a subject diagnosed with preeclampsia;

FIG. 3 is a flowchart showing a workflow including training a machine learning procedure in accordance with embodiments;

FIG. 4 is a flowchart showing a workflow including applying a machine learning procedure in accordance with embodiments, and

FIGS. 5 to 9 are tables of results;

FIG. 10 is a flowchart showing in overview a method for training a machine learning procedure in accordance with an embodiment;

FIG. 11 is a plot of performance results obtained for a machine learning procedure trained using the method of FIG. 10;

FIGS. 12 to 17 are tables of further results and data.

### DETAIL DESCRIPTION

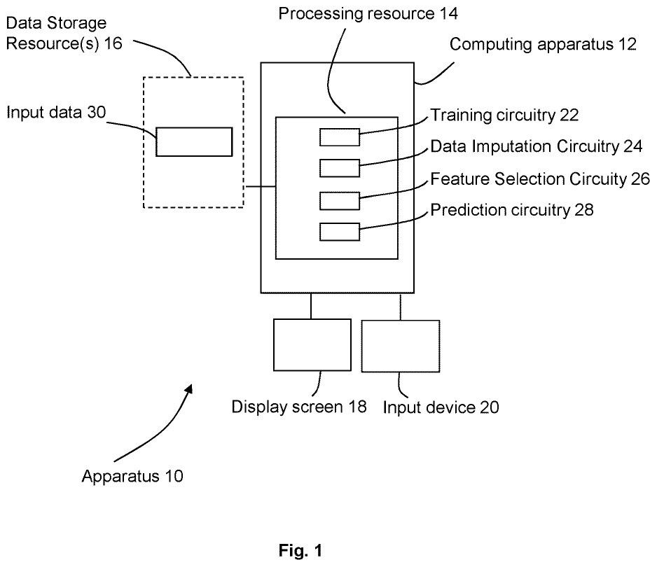

FIG. 1 is a schematic diagram depicting a computing apparatus 10 for performing a method of obtaining a risk level and/or a probability associated with one or more adverse maternal outcomes for a subject with suspected or confirmed preeclampsia, in accordance with embodiments.

FIG. 1 depicts a computing apparatus 12. The computing apparatus 12 has a processing resource 14, one or more data storage resources 16, a display screen 18 and an input device 20.

It will be understood that while FIG. 1 depicts a single computing apparatus for the purposes of the following description, the computing apparatus may be a distributed computing apparatus. For example, the computing apparatus 12 may comprise two or more computing apparatuses over a network. Likewise, the one or more data storage resources may be distributed data storage resources, for example, data may be retrieved from databases over a network.

The computing apparatus 12 comprises a processing resource 14. In the present embodiment, the processing resource 14 comprises a Central Processing Unit (CPU). For the purposes of the following description, the processing resource 14 has training circuitry 22, data imputation circuitry 24, feature selection circuitry 26 and prediction circuitry 28. The prediction circuitry may also be referred to as classification circuitry. While each of these circuitries is depicted in the processing resource 14, it will be understood that in some embodiments, one or more of these circuitries may be provided as part of a further processing resource, for example, of a network connected computing resource. In particular, it will be understood that training may be performed on a further computing resource. In some embodiments, the training circuitry may be implemented on a dedicated processing unit, for example, a GPU. It will be understood that, in some embodiments, processing steps can be performed on a combination of computer processing units (CPUs) and graphics processing units (GPUs) and other dedicated processing units. As an example, prediction including classification data processing may be performed separately to other data processing steps, such as training, imputation and feature selection. As such, a separate computing apparatus, for example, a portable computing device, may be provided to receive user input and perform the prediction steps based on the received user input.

The circuitries may also be referred to, in some embodiments, as modules such that the apparatus has a training module configured to train a model or procedure, data imputation module configured to perform a data imputation process, feature selection module configured to perform a feature selection process and prediction module configured to apply a prediction module.

In the present embodiment, the various circuitries of the processing resource 14 are each implemented in the processing resource 14 by means of a computer program having computer-readable instructions that are executable to perform the method of the embodiment. However, in other embodiments each circuitry may be implemented in software, hardware or any suitable combination of hardware and software. In some embodiments, the various circuitries may be implemented as one or more ASICs (application specific integrated circuits) or FPGAs (field programmable gate arrays).

The computing apparatus 12 also includes a hard drive and other components including RAM, ROM, a data bus, an operating system including various device drivers, and hardware devices including a graphics card. Such components are not shown in FIG. 1 for clarity.

Turning to the data storage resources 16, for the purposes of the description, FIG. 1 depicts the data storage resources 16 having a number of different storage modules. The data storage resource 16 may include a hard drive or other suitable memory resource, for example, a network connected database. It will be understood that the data storage resources 16 may be a network distributed storage resource.

As described in the following, the data storage resources 16 stores input data 30 for use during training, data processing and/or prediction data processing steps. The data storage resources can also provide a storage resource for trained model data, in some embodiments. In particular, trained model parameters and model architecture parameters. In some embodiments, one or more of these types of data has a corresponding data storage resource and the processing resource is configured to perform the required data operations on the corresponding resource as required.

The display screen 18 may also be referred to as the display, for brevity. The input device 20 is configured to receive user input data representative of a user input. The user input and display may be considered to form a user interface. The display screen 18 may display a graphical user interface for a user to interact. The input device may also be referred to as a user input device.

It will be understood that while FIG. 1 depicts an apparatus for training a procedure and for applying the trained procedure, in some embodiments, the training process will be performed separately to the application of the trained procedure. In such embodiments, learned procedure parameters, such as weights and/or coefficients may be retrieved from local storage and/or over a network or any suitable data communication interface.

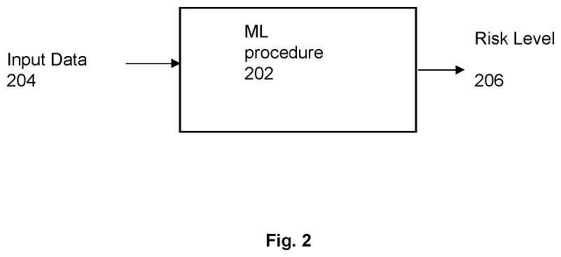

FIG. 2 is a schematic diagram depicting a machine learning derived procedure 202 for obtaining a risk level or probability 206 for one or more adverse maternal outcomes for a subject with suspected or confirmed preeclampsia. As described in further detail in the following, the machine learning derived procedure is obtained by performing a training process on one or more data sets. The machine learning derived procedure may refer to or be dependent on a trained machine learning model and the machine learning derived procedure may comprise applying the trained model to input data. The machine learning derived procedure may be dependent on a set of trained model weights or model coefficients and the procedure may comprise a set of rules or instructions for combining the trained model weights or model coefficients to input data.

In the embodiments descried in the following, an embodiment in which a value for at least one statistic associated with the health care system and/or a country and/or a region of the health care system is provided as part of the input data to the machine learning procedure is described. Such embodiments may offer advantages for example, these embodiments may prevent overfitting of a model to local outcome rates. In addition, such embodiments may allow the model to offer predictions in healthcare settings where obtaining certain types of subject data may be more problematic.

In addition, embodiments in which a reduced set of features is used to obtain a reduced set of input variables from a larger set of input variables are described. There may be advantages relating to using fewer data for obtaining predictions, such as fewer resources required, including both health care resources and processing resources.

In addition, embodiments using dynamic models are described in the following. Such models may offer advantages in a health care setting, as good performance may be maintained over a length of stay of a patient.

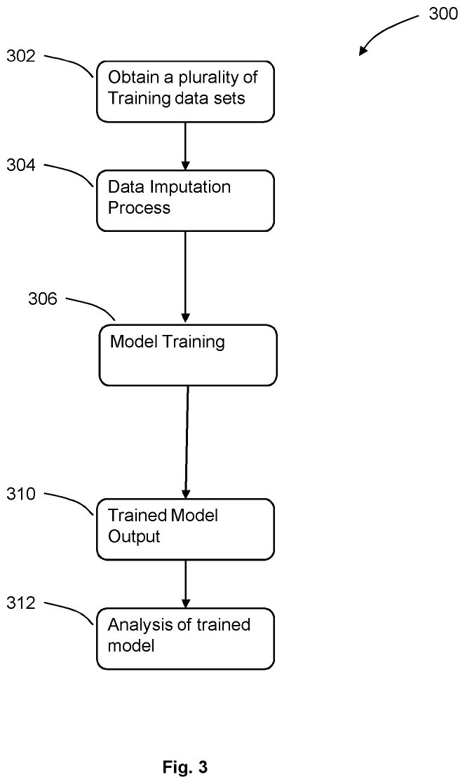

In the present embodiments, the machine learning derived procedure can be considered as a set of learned rules and/or instructions for operating on input data. In the present embodiments, the machine learning procedure corresponds to a set of rules and/or a procedure for classifying a set of received input data representative of a plurality of parameters as a risk level, where the risk level of the occurrence of one or more adverse maternal outcomes for a subject with suspected or confirmed preeclampsia. The risk level may be a risk level from one of a group of classes: very low, low, moderate, high, very high. In the present embodiment, the machine learning derived procedure is a machine learning classifier, and may be referred to, for brevity, as a classifier. The classifier is a trained classifier and the training is described with reference to, for example, FIG. 3.

For a subject, for example a patient that has been diagnosed with suspected or confirmed preeclampsia, input data 204 are obtained and are provided to the trained procedure. In the present embodiment, the input data 204 are representative of values for a set of 18 input variables. These 18 variables include: platelet count (×10⁹ per litre); Haematocrit (%); aspartate transaminase (U/L); alanine transaminase (U/L); uric acid (μ/L); total leukocyte count (×10⁹ per litre); serum creatinine (μM); Gestational age at eligibility (wk); weight at eligibility (kg); mean platelet volume (fL); height (cm); diastolic blood pressure (mm Hg); maternal age at expected date of deliver (yr); serum albumin (g/L); national per capita Gross Domestic Product (GDP) (US dollars); oxygen saturation (SpO₂—Oxygen saturation by pulse oximetry) and national maternal mortality ratio (MMR) (per 100,000 live births). GDP per capita is defined according to a https://data.worldbank.org/indicator/NY.GDP.PCAP.CD. MMR per capita is defined at https://databank.worldbank.org/reports.aspx?source=world-development-indicators #. In some embodiments, the most informative maternal mortality ratio (e.g. one of national or regional) may be used.

While the present embodiment is directed to procedure trained to receive input data representing values for a set of 18 parameters and output a risk level based on these 18 parameters, the set of 18 input variables are derived using a model development and training process, as described in further detail with reference to FIG. 3. It will therefore be understood that, in alternative embodiments, a procedure that receives values for an alternative set of input variables for a subject selected may be trained to classify a risk level.

In the embodiments described above, a classifier that classifies received input data in terms of a risk level is described. In the present embodiment, the output of the classifier is a risk level associated with a Delphi-derived composite outcome of maternal mortality or severe morbidity within two days of first assessment with pre-eclampsia. In the present embodiment, the outputted risk level is one of five risk levels: “very low”; “low”; “moderate”, “high”, “very high”. However, it will be understood that in other embodiments, the procedure is a machine learning derived procedure that can output alternative output. In other embodiments, the output from the procedure may be a predicted probability for a specific maternal outcome and/or a score or percentage representing the risk level.

In general, the input data provided as input to the trained classifier is representative of values of input variables. The input data include subject data selected from at least two groups that can be referred to as: demographic data; vital sign data representative of one or more vital signs of the subject, and sample data representative of one or more parameters obtainable from an analysis data. In the present embodiment, the sample data includes platelet count, haematocrit, aspartate transaminase, alanine transaminase, uric acid, total leukocyte count, serum creatinine, mean platelet volume, weight at eligibility, serum albumin; the vital sign data includes weight at eligibility, oxygen saturation, height, diastolic blood pressure; the demographic data includes Gestational age at eligibility, maternal age at expected date of delivery. In the present embodiment, the input data also includes healthcare system data. The healthcare system data includes one or more statistics or indicators associated with the country or region of the healthcare system for the subject. In the present embodiment, the healthcare system data includes national per capita Gross Domestic Product and the most recent estimation of the national maternal mortality ratio.

The vital sign data and demographic data may be considered together as representing patient data. The sample data is representative of one or more parameters obtainable from an analysis of a sample. The sample data and vital signal data may be considered as examples of clinical data. The healthcare system data may include at least one statistic or indicator that is non-clinical, for example, national per capita GDP.

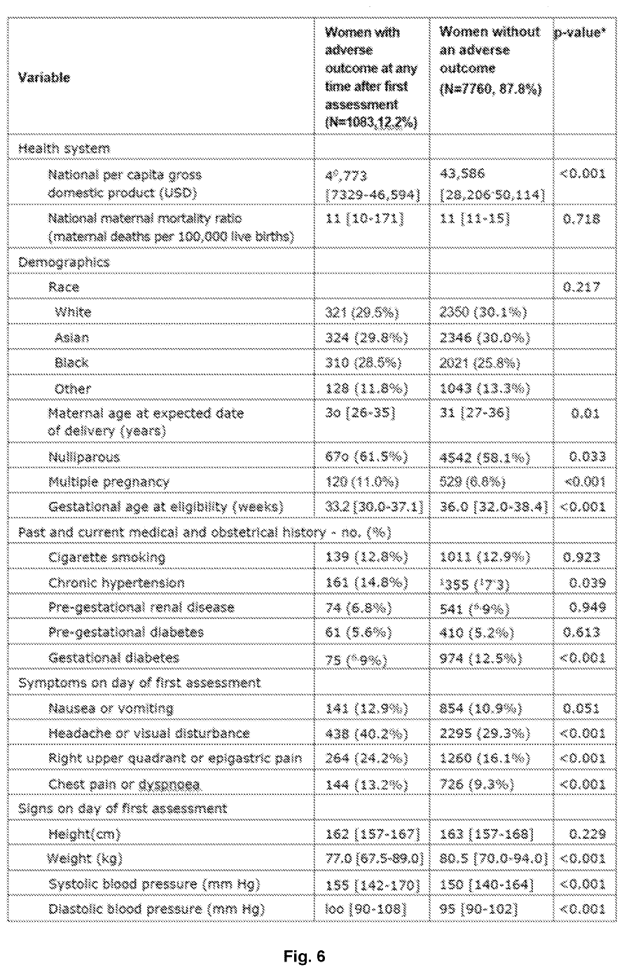

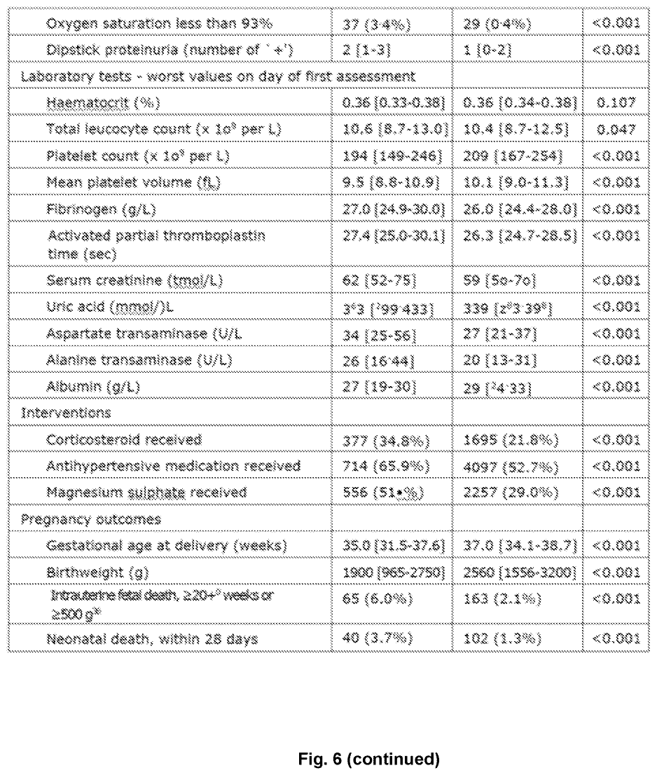

While different machine learning derived procedures may be trained, in the embodiments described in the following the procedure is a random forest based procedure. In the present embodiment, the features used to train the classifier are the features depicted in FIG. 6. The classifier, once trained, may be used to classify new input data representative of a new subject, for example, by performing the method of FIG. 3.

FIG. 3 depicts an example method of training and developing a machine-learning procedure, in accordance with embodiments.

At step 302, a plurality of data sets are obtained. The plurality of data sets are combined into a combined dataset, which is then grouped into: a development data set (containing development data); a validation data set (containing validation data) and a further group for selecting cut-off points for the risk groups. In the present embodiment, the development data set was 75% of the combined data; the validation data set was 12.5% of the combined data, and the further group for selecting cut-off points for the risk groups was 12.5%. The present method uses multiple risk groups for selecting the cut-off and reporting the performance. The cut-offs were selected based on likelihood ratios.

The plurality of data sets includes data sets from model development and validation studies for: (i) miniPIERS: N=2081 women from Brazil, Fiji, Pakistan, South Africa, and Uganda; 5 (ii) fullPIERS development: 8 N=2023 women; Australia, Canada, New Zealand, and the United Kingdom; and (iii) fullPIERS validation: 11 Canada [N=1310 women, single centre (Vancouver)], Finland [N=124 women, multicentre], 12 United Kingdom [N=1101 women, multicenter; 13 N=70 women, multicentre; 14 and N=281 women, single centre], and N=644 women, Canada and the United States). The training data was prospectively-collected published data from women with broadly-defined pre-eclampsia as they presented for initial facility-based assessment at centres with general policies of expectant management remote from term. It will be understood that suspected or confirmed pre-eclampsia may have different definitions. In the present embodiment, pre-eclampsia was defined as: gestational hypertension accompanied by one or more of the following new-onset conditions at ≥20 weeks gestation: 1) Proteinuria; 2) Other maternal end-organ dysfunction, including: i) Neurological complications (e.g., eclampsia, altered mental status, blindness, stroke, clonus, severe headaches, or persistent visual scotomata), ii) Pulmonary oedema, iii) Haematological complications (e.g., platelet count<150,000/μL, DIC, haemolysis), iv) acute kidney injury (such as creatinine≥90 μmol/L or 1 mg/dL), v) Liver involvement (e.g., elevated transaminases such as alanine transaminase or aspartate transaminase>40 IU/L) with or without right upper quadrant or epigastric abdominal pain); 3) Uteroplacental dysfunction (e.g., placental abruption, angiogenic imbalance, fetal growth restriction, abnormal umbilical artery Doppler waveform analysis, or intrauterine fetal death). Among women with chronic hypertension, pre-eclamspia was defined as development of new proteinuria, another maternal organ dysfunction(s), or evidence of uteroplacental dysfunction (as above).

The training data may be further enriched with inclusion of PREP data representing women with early-onset preeclampsia (diagnosis at less than 34 weeks).

At step 304, a data imputation process is performed to compensate for missing and/or duplicated data. In the present embodiment, the data imputation step was performed in accordance with a set of predefined rules. For example, one rule that was applied is: if multiple measurements per woman are available for a variable then the multiple measurements are filtered so that the worst measurement per day was included. The imputed data then includes a data point corresponding to only one observation for that variable, per woman per day. In the present embodiment, the worst measurement corresponds to “Yes” for symptoms of headache or visual disturbance, nausea or vomiting, right upper quadrant or epigastric pain and chest pain or dyspnoea; as the Minimum for oxygen saturation, platelet count, fibrinogen and serum albumin; and as the Maximum for systolic and diastolic blood pressure, total leucocyte count, mean platelet volume, haematocrit, activated partial thromboplastin time, serum creatinine, uric acid, alanine transaminase, aspartate transaminase and dipstick proteinuria.

For any missing data, these were handled based on the mechanism of missingness and the degree of missingness. The handling of missing data is in accordance with a missing data replacement scheme. In the present embodiment, data were excluded from the analyses if the determined missingness was greater than or equal to 60%. Missing values for variables with less than 60% missingness were dealt with by using multiple imputation.

The mechanism of missingness may be missing completely at random (MCAR) when the missingness is completely by chance, and isn't related to any observed or unobserved data. The mechanism of missingness may be missing at random (MAR) when missingness is related to some observed data but there is no relation between the observed and the unobserved data. The mechanism of missingness may be missing not at random (MNAR), where the unobserved data determines the missingness.

In the present embodiment, as approximately 19% of all training data was missing, the development data are imputed using all predictor variables separately to the validation data, 20 times based on a rule of thumb that the number of imputations should be at least the percentage of incomplete cases or more, using chained random forests to minimize bias. This step is a pre-processing step, used on the data to fill in the missing values, before model development. The predictor variables are restricted to variables with above average importance using the Gini index. The validation data are also imputed independently once, using the same method.

In the present embodiment, oxygen saturation was imputed. In an alternative embodiment, missing values of oxygen saturation are replaced by the value “97%”. This value is selected based on the assumption that they were ‘missing not at random,’ as oxygen saturation was less likely to be measured if thought to be normal. As a result of the data imputation process, a number of imputed data sets are made available for a model development process.

By imputing missing data, potential problems associated with replacement of missing values with a value that do not appear in the data may be avoided. For example, methods such as random forests and gradient boosted trees operate by essentially passing data through a series of questions/tests. For example, if a missing blood pressure value was replaced by a “−1” and one of the tests, for example, of the random forest, was “blood pressure <100 means less likely to have a certain outcome”, all subject with a missing value would be classed as “less likely”.

The model development process includes steps of training one or more models 308 and/or performing a feature selection process 310. While these steps are depicted as two separate steps, it will be understood that, in some embodiments, the feature selection process forms part of the training process and vice versa. For example, in the present embodiment, random forests are trained and parameters derived or related to the random forests are used as part of the features selection process.

Variables are considered for modelling only if they had been assessed prior to the first occurrence of a component of the combined adverse maternal outcome. Any data that is not assessed prior to occurrence of a component of the combined adverse maternal outcome is discarded before making the training, testing and validation datasets, not counted in any of the reported numbers. The following set of variables are used for the training process: health system (national per capita gross domestic product; maternal mortality ratio for country of residence and year of publication); demographics (self-declared ethnicity; gravidity; parity; singleton or multiple pregnancy; maternal age at expected date of delivery; gestational age at assessment); past and current medical and obstetrical history (cigarette smoking; number of living children; gestational diabetes in index pregnancy; pre-gestational diabetes, renal disease, or hypertension); symptoms (headache/visual disturbance; chest pain/dyspnoea, nausea/vomiting; right upper quadrant/epigastric pain); signs (height and weight at assessment; systolic and diastolic blood pressure; oxygen saturation; dipstick proteinuria); and laboratory tests (haematocrit; total leukocyte count; platelet count; mean platelet volume; serum creatinine; uric acid; albumin; alanine and aspartate transaminase; lactate dehydrogenase; total bilirubin). Oxygen saturation (marker of cardiorespiratory and non-cardiorespiratory outcomes), uric acid (inflammation, oxidative stress, decreased renal clearance, and risk of adverse maternal and foetal outcomes), lactate dehydrogenase (haemolysis and transaminitis) were evaluated. For face validity, inclusion of at least one objective variable was required for each of cardiorespiratory, renal, hepatic, and haematological organ systems. The list of variables are depicted in FIG. 6.

At step 306, in the present embodiment, an ensemble of random forest based models are trained using training data. Each random forest comprises a plurality of decision trees. As part of the training process, training parameters for the random forest parameters are selected, for example, the number of trees and/or number of variables to try at each split in the random forest.

At a first step, the training data is split into datasets and each dataset is used to train a corresponding random forest. A random forest is trained by constructing several decision trees during training. A random forest is fitted for each of the imputed datasets. When fitting the random forest, first a random subset of rows and variables is selected and a decision tree is built on this subset. Then a new subset is selected and a new tree is fitted, this part is repeated until the desired number of trees is reached. Each node of each tree corresponds to a variable under consideration. Each trained decision tree thus provides a non-linear relationship between the input data and the output. For the random forest, the output of the random forest is the mean (or other weighted sum) of the output of all trees of the forest.

Any suitable random forest training techniques may be used, for example, methods used and described in “Random forests”, by Breiman L., Mach Learn 2001; 4(1): 5-32; Brownlee J. Classification and regression trees for machine learning. 15 Aug. 2020 2016, as found at https://machinelearningmastery.com/classification-and-regression-trees-for-machine-learning/.

Following the training, the trained random forest based models are then passed to the feature selection process 310. In the present embodiment, the feature selection process 310 includes applying an above average importance algorithm to identify the most important variables, or a specified number of the most important variables. Other variable selection methods may be used (including recursive feature elimination to identify and eliminate the least important variables, Vita and Boruta methods). In the present embodiment, the most important 18 features were determined using the feature selection process. It will be understood that the feature selection process may also be referred to as a variable selection process.

It will be understood that the scores, such as importance parameters and/or other derived metrics from the training process are used for the feature selection process. In the present embodiment, feature elimination is performed using a random forest model. In the present embodiment, 10 imputed data sets were used to train a corresponding 10 models and 10-fold cross validation was then used to fit the models. The mean decrease in Gini index was used to obtain variable importance, according to which the variables were ordered. Using this technique, the 18 variables with the highest importance scores were selected using an above average importance selection algorithm.

Following the feature selection process 308, the method returns to step 306 to train one or more further procedures using the selected subset of variables, which, in this embodiment, comprises the selected 18 variables. While the present method includes a feature selection process, it will be understood that the feature selection process is an optional step.

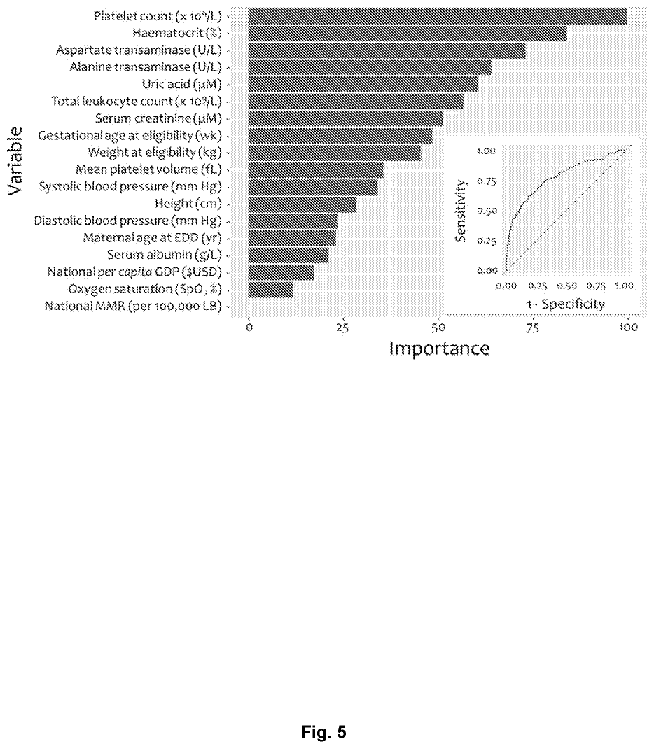

FIG. 5 depicts the 18 variables selected and used for the final trained model. The variables are ranked by importance within the random forest model based on Gini index, compared with the least important variable (National MMR). The variables are: platelet count (×10⁹ per litre); Haematocrit (%); aspartate transaminase (U/L); alanine transaminase (U/L); uric acid (u/L); total leukocyte count (×10⁹ per litre); serum creatinine (uM); Gestational age at eligibility (wk); weight at eligibility (kg); mean platelet volume (fL); height (cm); diastolic blood pressure (mm Hg); maternal age at expected date of deliver (yr); serum albumin (g/L); national per capita Gross Domestic Product (GDP) (US dollars) oxygen saturation (SpO₂—Oxygen saturation by pulse oximetry) and national maternal mortality ratio (MMR) (per 100,000 live births).

In other embodiments, other feature selection processes may be used. Other feature selection process may results in a different subset and/or number of selected features.

These selected variables do not include symptom data that is representative of one or more symptoms of the subject. Such variables are not selected as they do not substantially improve predictive performance after the first 18 variables were used. The selected variables include data representative of national per capita gross domestic product, maternal mortality ratio, systolic and diastolic blood pressure, uric acid.

The inset of FIG. 5 is a plot of the area under receiver operator characteristic for adverse maternal outcomes within two days of initial assessment, using data within one day of initial assessment and prior to the occurrence of any outcome.

As described above, random forest methods enable examination of feature importances, which is the mean of the amount the Gini Index (or node impurity) decreases by in each tree at the split which uses the feature. The more the Gini Index decreases for a feature, the more important it is. This figure rates the features from 0-100, with 100 being the most important. Area-under-the receiver-operator characteristic is calculated as 0.78 [95% Cl 0·73, 0·82]. The importance scores can then be used to inform the feature selection process.

At step 310, data representative of the trained procedure is stored to allow the trained procedure to reconstructed for use on new input data. The stored data includes the trained model parameters (for example, the splits within the trees of a random forest and their location).

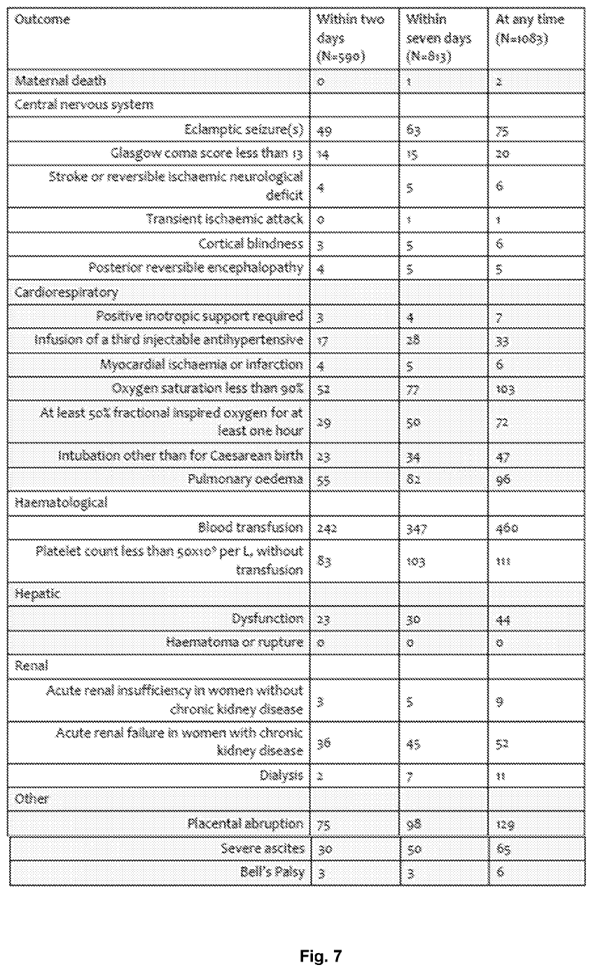

At step 312, an analysis process using the trained model was performed using the validation data. Further detail on the analysis and results is provided with reference to FIGS. 7 to 9.

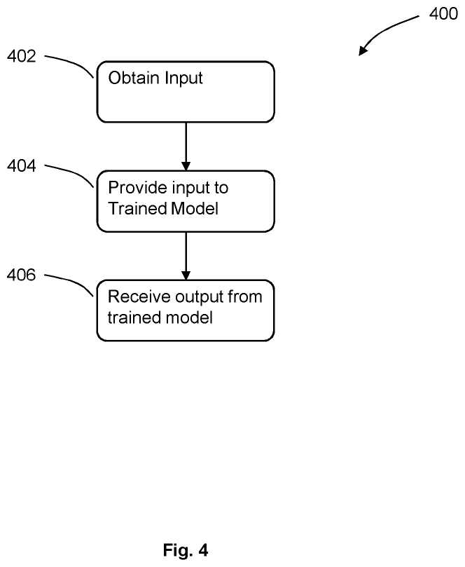

FIG. 4 depicts a workflow of a method of obtaining adverse maternal outcome information for a subject. At step 402, input is obtained. In the embodiment of FIG. 4, the display screen 18 displays prompts for the user input parameters. The display screen displays a graphical user interface, for example, via an internet browser or other suitable interface. In some embodiments, one or more controllable display elements, such as drop down menu elements and/or toggle element are presented on display screen. In some embodiments, the display screen 18 may display a form for providing numerical input. In the present embodiment, user input data is generated in response to user input being received at input device 20. The user input data is representative of values for the input data. For example, in embodiments, in which a selected set of input variables is used, the user input data may represent values for the set of input variables. In other embodiments, the input may be provided in the form of a suitable data file. The user input may be provided in any suitable format, such as numerical input.

At step 404, the received input data is provided to the trained machine learning procedure, for example, the machine learning procedure described with reference to FIG. 2 and trained as described with reference to FIG. 3.

At step 406, the trained machine learning procedure operates on the received input data and calculates an output. In this embodiment, the machine learning procedure is a trained classifier as described with reference to FIG. 2 and the output is a classification of risk level. In this embodiment, the output is displayed on display screen 18.

In the above-described training and development process, a machine learning derived procedure configured to receive input representing 18 variables was described. However, it will be understood that, in other embodiments, alternative machine learning procedures may be trained that use alternative sets of variables. In a further embodiment, the training and development process includes performing a feature selection process using the development data to select an alternative subset of the variables and training a machine learning procedure to operate on the alternative subset of the variables.

In a further embodiment, recursive feature elimination was performed using random forest model. For each imputed data set, of which there were 20, a recursive feature elimination R function was used to train a model. 10-fold cross validation was then used to order the variables on their importance. The top 8 variables with the highest importance score were extracted from each into a plurality of importance lists. In this embodiment, the number 8 was selected as it was found that model with 8 variables had an accuracy that was only slightly improved by using more than 8 variables.

In this embodiment, four variables (oxygen saturation, platelet count, aspartate transaminase, alanine transaminase) appeared in the top 8 variables for all 20 of the generated lists of important variables. The two variables serum creatinine and symptom of chest pain or dyspnoea appeared in 19 of the generated lists. The variable haematocrit appeared in 18 of the generated lists. The variable symptom of right upper quadrant or epigastric pain appeared in 13 of the generated lists. The variable uric acid appeared in 9. The two variables serum albumin and symptom of vomiting or nausea appears in 1 out of the 20 lists. The combination of the following 11 variables were selected in this embodiment: oxygen saturation, platelet count, aspartate transaminase, alanine transaminase, haematocrit, serum creatinine, serum albumin, uric acid, chest pain or dyspnoea, symptom of right upper quadrant or epigastric pain, and symptom of vomiting or nausea.

It will be understood that, aside from albumin, all the other blood test variables for this 11 variable model are among the blood tests most commonly available. As this model requires fewer variables, and some of those variables are symptoms, it may offer advantages for use in lower to middle income settings.

Training data representative of the selected, reduced parameter set was then used to train a further machine learning procedure that receives input data representing only these data. In the present embodiment, the further machine learning procedure is a random forest based procedure, as described with reference to FIGS. 2, 3 and 4.

In the above-described embodiments, a random forest based process was described. However, it will be understood that, in further embodiments, alternative machine learning models may be trained. For example, LASSO and ridge regression, artificial neural networks and Bayesian Model averaging may be used.

In the above-described embodiments, input data representative of values for a number of input variables are obtained. In some embodiments, the method include obtaining one or more of these parameters by performing a sampling process, for example, obtaining a sample, such as a fluid sample, from a subject and performing a sample analysis on the sample. The sample may be a blood sample and wherein the sample analysis may be an analysis to obtain at least one of: a) one or more haematological parameters including haematocrit, platelet count, total leukocyte count; b) one or more renal parameters including lactate dehydrogenase, aspartate transaminase, alanine transaminase, serum albumin. In embodiments in which health care system data is used, the method may include obtaining user input representative of the country and/or region of the subject and retrieving the desired value of the health care system variable.

In some embodiments, applying the trained model includes applying an ensemble of more than one trained model to input data.

In some embodiments, a data structure is constructed that has a form for input to the trained procedure and the data structure is populated using the input data, for example, using input data obtained from a data storage resource and/or from user input. In the present embodiment and as described in further detail in the following, the procedure is applied to the input data to output a risk level/probability associated with one or more adverse maternal outcomes for a subject.

In some embodiments, applying the procedure includes the step of applying a trained model to the data structure to generate a further data structure representative of a probability/risk level. Generating a further data structure may include, for example, constructing the further data structure for a subject, applying the trained model to the input data structure to populate the further data structure with values representative of risk level and/or probability for the subject.

In contrast to known methods, the present embodiment uses a maternal outcome only, rather than a composite maternal and foetal outcome. Using maternal outcomes only reduces any complication from composite outcomes (for example, due to the maternal risk increasing over time and the foetal risk decreasing). In addition, providing an outcome representing a risk level for only a maternal outcome may be more useful for making a decision than a composite outcome. Showing that someone is at a high risk for a maternal or a foetal outcome may not be as useful for making a decision as providing risk estimates for the two separately.

In the above-described embodiments, a random forest model is described. In the following described embodiment, input data including subject data obtained over a period of time, for example, longitudinal data representing repeated observations for a subject over a period of time are used as training data for training a dynamic model. In this embodiment, the input data is time dependent in that for each variable, multiple observations are available and used for training. As described in the following, such models may be used to obtain predictions at successive points in time, for example, at least at two different times during a time period. For example, a prediction of risk level and/or probability could be made at every consecutive day from the first day of admission for a patient. At least part of the subject data may be referred to as longitudinal data in that the data represents repeated observations of the same variables from the same subjects over a period of time.

In the following, the term dynamic is used to contrast such models against so-called static models that may be trained, and tested, on a single set of observations. While such static models may be used at different time points, and can be used repeatedly on multiple, repeated observations to create new predictions using the latest available set. This is referred to as consecutive prediction. Advantages of dynamic models for consecutive prediction may include accounting for the magnitude and rate of change in the predictor variables over time. Moreover, as static models are usually only tested for the set of observations from a pre-defined timepoint (for example the first or last observations), a maintained performance over time may not be guaranteed. Static models may require to be tested for consecutive prediction and recalibrated at different timepoints as appropriate, or other methods should be considered where including multiple observations at a time is a possibility. In the following description, the term variable may refer to predictor variables or target variables. A predictor variable is a variable whose values will be used to predict the value of the target variable. Dynamic modelling may cover statistical or machine learning procedures trained using longitudinal training data to predict an outcome.

In further detail, the data used for training the dynamic model was combined from previously published studies and split into development and validation datasets for the modelling. Where multiple measurements were available per patient per day, the worst measurement per day was taken. Missing data was imputed using multiple imputation.

As described with reference to FIG. 2, the development and validation datasets were imputed separately. In the following, the time input to the model corresponds to an elapsed time from a pre-defined clinical event. In the present embodiment, the clinical event is admission of the subject to a health care setting. However, the clinical event may be a different event, for example, an occurrence of a symptom or another measurable property reaching a threshold.

A number of different dynamic modelling methods were developed and tested. These include likelihood contrast, two stage models of generalised linear mixed effects models

(GLMMs) and logistic regression, joint models of GLMM and logistic regression, Binary Mixed Model (BiMM) forests, which combine Bayesian GLMMs and random forests, and recurrent artificial neural networks.

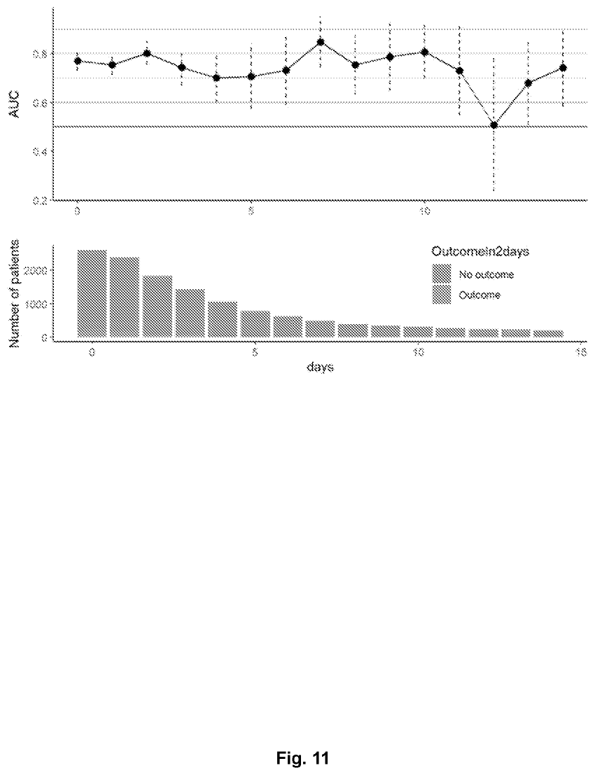

The best performance over time was achieved by BiMM forest based model. FIG. 11 is a plot of a measure of performance. FIG. 11 depicts the estimated area under the receiver operator curve (AUC). This is above 0.7 for the first 11 days after admission. The other methods performed similarly to assigning patients to outcome groups by chance, with AUCs between 0.5 and 0.6 for all models and days.

Binary Mixed Model (BiMM) forests combine Bayesian GLMMs with random forests. In such a model, fixed components are modelling using random forest and random components are modelled using a Bayesian GLMM part of the model. The BiMM forest is fitted through an iterative process. BiMM models were first explored in “BiMM forest: A random forest method for modeling clustered and longitudinal binary outcomes”, Chemometrics and Intelligent Laboratory Systems. 2019; 185:122-34 by Speiser et al.

The training of the BiMM forest based procedure iterates between developing random forest models using all predictors and then using information from the random forest model within a Bayesian GLMM to account for the clustered structure of the outcome. In the present embodiment, the BiMM forest was tested with all predictors, and with manual selecting only the variables that were used in the 18 variable static model, as described above, for example, with reference to FIG. 3. However, it will be understood that, in alternative embodiments, a separate variable selection process may be performed for the BiMM forest based procedure.

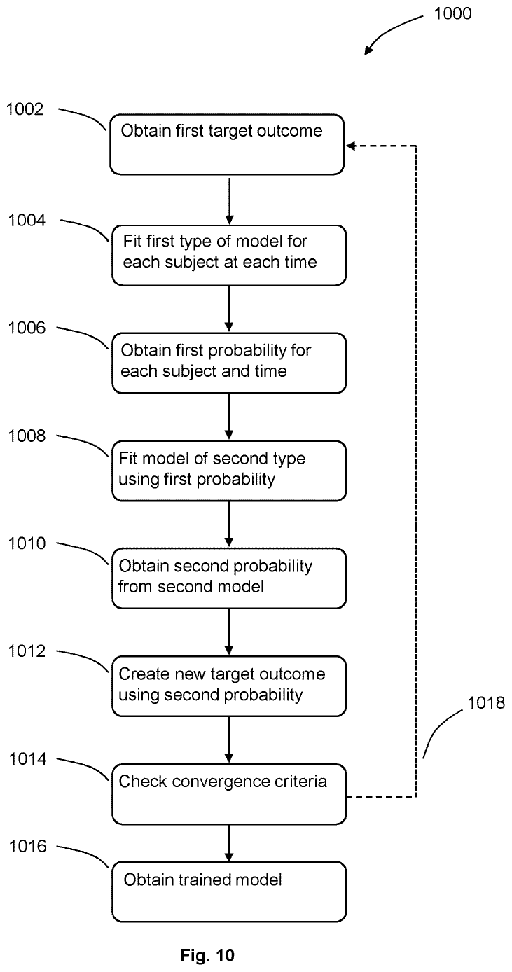

The iterative training process for a dynamic machine learning procedure is described with reference to FIG. 10. It will be understood that the process of FIG. 10 can be implemented in a workflow such as that described with reference to FIG. 3. For example, prior to the training steps, it will be understood that input data is obtained and, optionally imputed, as described with reference to FIG. 3. However, in contrast to FIG. 3, the input data for each subject corresponds to input data obtained for a plurality of time points. At a first step 1002, a model of a first type, in this embodiment, a random forest, is fitted using the fixed predictors (Xᵢₜ) for each patient (i=1, 2, . . . , N) at each timepoint (t=1, 2, . . . , Tᵢ), using yᵢₜ, the binary outcome, as the outcome of the model. Xᵢₜ represents the input data for the ith patient at the tth time point. Xᵢₜ may be represented in any suitable mathematical representation, for example, This stage of the model fitting considers each observation per patient as a separate data point and therefore does not take previous observations per patient into account.

At a second step 1004, a predicted probability for each patient and time point is extracted from the random forest. The probability from the random forest for each patient and time point is represented as p_{RF}(Xᵢₜ).

At a third step 1006, a model of a second type, in this embodiment, a Bayesian GLMM is fitted as follows:

$$logit ( {y}_{i t} ) = {β}_{0} + {β}_{1} * {p}_{R F} ( {X}_{i t} ) + {Z}_{i t} * {b}_{i t}$$

In the above equation, Zᵢₜ is a clustered variable and bit is the random effect due to, for example, a patient. p_{RF}(Xᵢₜ) is the predicted probability from the random forest of each longitudinal observation t=1, . . . . Ti, for each patient/cluster i=1, . . . . N. In the above formula, βᵢ are the coefficients of the logistic regression model, β₀ being the coefficient for the intercept and β₁ the coefficient of p_{RF}(Xᵢₜ).

At a fourth step 1010, a second predicted probability for each patient and time point is extracted from the Bayesian GLMM, represented as p_{BGLMM}(Xᵢₜ, Zᵢₜ).

As a fifth step 1012, a new target outcome

$${y}_{i t}^{*}$$

is created by adding p_{BGLMM}(Xᵢₜ, Zᵢₜ) to the previous target outcome yᵢₜ for each i and t. As yᵢₜ is a binary value taking either a 0 or a 1 value, and p_{BGLMM}(Xᵢₜ, Zᵢₜ) is a probability between 0 and 1, adding the two together creates a continuous numeric value between 0 and 2. To convert the sum into a binary value, a predefined split function h( ) is applied, as follows:

$${y}_{i t}^{*} = h ( {y}_{i t} + {p}_{B G L M M} ( {X}_{i t} , {Z}_{i t} ) )$$

At the next step 1014, a convergence criteria is checked to determine if the model is sufficiently trained. In the present embodiment, the criteria is an assessment of a posterior log likelihood from the Bayesian GLMM being less than a prespecified value.

If the convergence criteria is satisfied, the method proceeds to step 1016, at which point the mode is considered sufficiently trained. The iterative process is then stopped, and no new target outcome is created.

If the convergence criteria is not satisfied, the method returns to step 1002, and the target outcome yᵢₜ is replaced by a new target y*ᵢₜ outcome. Steps 1002 to 1014 are repeated until the posterior log likelihood from the Bayesian GLMM is less than a prespecified value. A skilled person will appreciate that variations of the enclosed arrangement are possible without departing from the invention. Accordingly, the above description of the specific embodiments are made by way of example only and not for the purposes of limitations.

The following non-limiting comments on an associated study and results relating to the above-described embodiments are provided for completeness.

Affecting two-to-four percent of pregnancies, pre-eclampsia is a leading cause of maternal death and morbidity worldwide. Machine learning was used to develop and validate a novel pre-eclampsia time-of-disease model with strong diagnostic test properties to rule-out and rule-in adverse maternal outcomes, while accounting for clinical setting. Health system, demographic, and clinical data from the day of first assessment with pre-eclampsia was used to predict a Delphi-derived composite outcome of maternal mortality or severe morbidity within two days, combining published data from 11 less- and more-developed countries. Machine learning methods, multiple imputation, and 10-fold cross-validation were used to fit models on a development dataset (3/4 data); validation was undertaken on the unseen quarter. Predictive risk accuracy was determined by area-under-the-receiver-operator characteristic (AU ROC), and risk categories were data-driven and defined by negative (−LR) and positive (+LR) likelihood ratios. Of 8843 participants, 590 (6.7%) developed the composite adverse maternal outcome within two days, 813 (9.2%) within seven days, and 1083 (12.2%) at any time. An 18-variable random forest-based prediction model—PIERS-AI—was accurate (AUROC 0.8 [0.76-0.84] vs fullPIERS: AUROC 0.68 [0.63-0.74]) and categorised women into very low (−LR<0-1, 0.7%), low (−LR 0.1 to 0.2, 29-1%), moderate (−LR>0-2 and +LR<5-0, 61.3%), high (+LR 5.0 to 10.0, 7.9%), and very high (+LR>10.0, 1.0%) risk; adverse maternal event rates were 0%, 2%, 5%, 25% and 64% within 48 hours, respectively. The PIERS-AI model improved identification of women with pre-eclampsia who are at lowest and greatest risk of severe adverse maternal outcomes within two days of assessment, and can support provision of accurate guidance to women, their families, and their maternity care providers.

Complicating two-to-four percent of pregnancies, pre-eclampsia (which may be defined. for example, as new-onset hypertension at or after 20 weeks gestation, accompanied by either new-onset proteinuria, other maternal target organ damage, or evidence of uteroplacental dysfunction) remains a leading, global cause of maternal mortality and life-threatening morbidity. 1-5 Over 99% of the annual 46,000 pre-eclampsia-related maternal deaths occur in low- and middle-income countries (LMICs). In pre-eclampsia pregnancies, perinatal survival without major morbidity is largely related to gestational age at birth.′ However, the burden of adverse maternal outcomes is spread across gestation; while the risks per woman are higher with preterm pre-eclampsia, the burden of maternal risk occurs at term. The sole method of initiating recovery is delivery of the placenta. At term, the focus is initiating birth. Before term, women and their maternity care-providers balance maternal risks from evolving disease with prematurity-related perinatal risks by subjectively integrating ongoing assessments of symptoms, signs, and laboratory tests. In busy maternity units, considerable experience informs decisions; however, most women with preterm pre-eclampsia are managed, at least initially, by maternity care-providers with less experience. For many LMIC and disadvantaged high-income country (HIC) populations, access to comprehensive obstetric and newborn care is limited. To optimise maternal outcomes in pre-eclampsia, objective, time-of-disease maternal risk assessment is needed to inform decision-making over the following 48 hours, wherever that woman lives. In the methods described, the strengths of machine-learning-based classifiers are used to develop and validate a globally-relevant PIERS-AI model, using information routinely-available at presentation with pre-eclampsia.

### Study Design and Included Datasets

Prospectively-collected data from women with broadly-defined pre-eclampsia was used, as they presented for initial facility-based assessment at centres with general policies of expectant management remote from term. To maximise the sample size for machine learning, data were collated from published model development and validation studies for: (i) miniPIERS (2008-2012): N=2126 women from Brazil, Fiji, Pakistan, South Africa, and Uganda; 5 and (ii) fullPIERS development and validation (2003-2016):812 N=6717; Australia, Canada, Finland, New Zealand, United Kingdom, and United States. A randomly-selected 75% of the combined cohort was used for model development, 12.5% were used to select cut-off points for risk strata, and 12.5% for model validation.

### Study Variables

Variables were considered for modelling only if assessed prior to the occurrence of any component of the combined adverse maternal outcome. Variables related to the woman's health system, and her demographics, past and current medical and obstetric history, symptoms, signs, and laboratory tests (Table of FIG. 6). For face validity, at least one objective variable was required for each of cardiorespiratory, renal, hepatic, and haematological systems.

### Study Outcomes

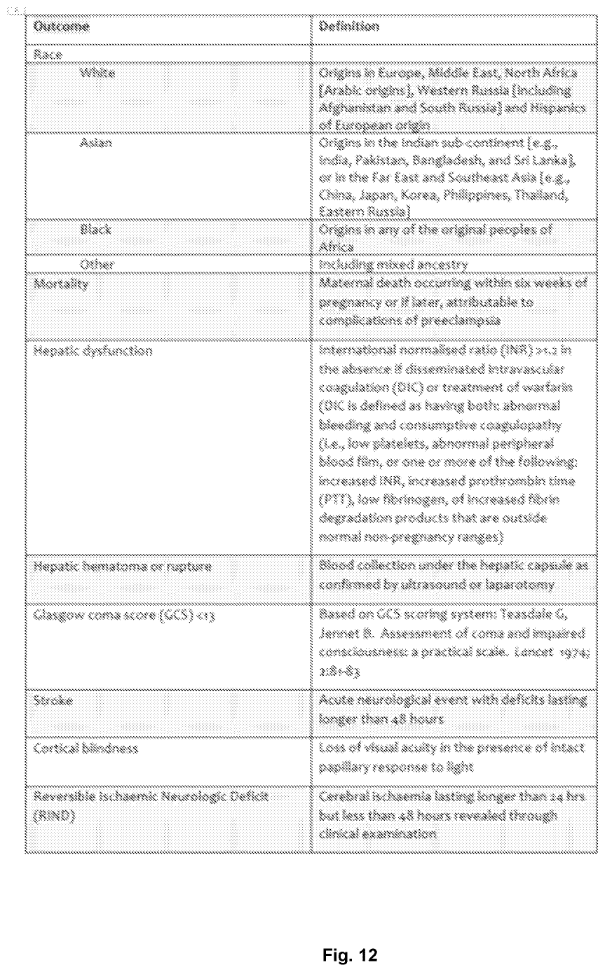

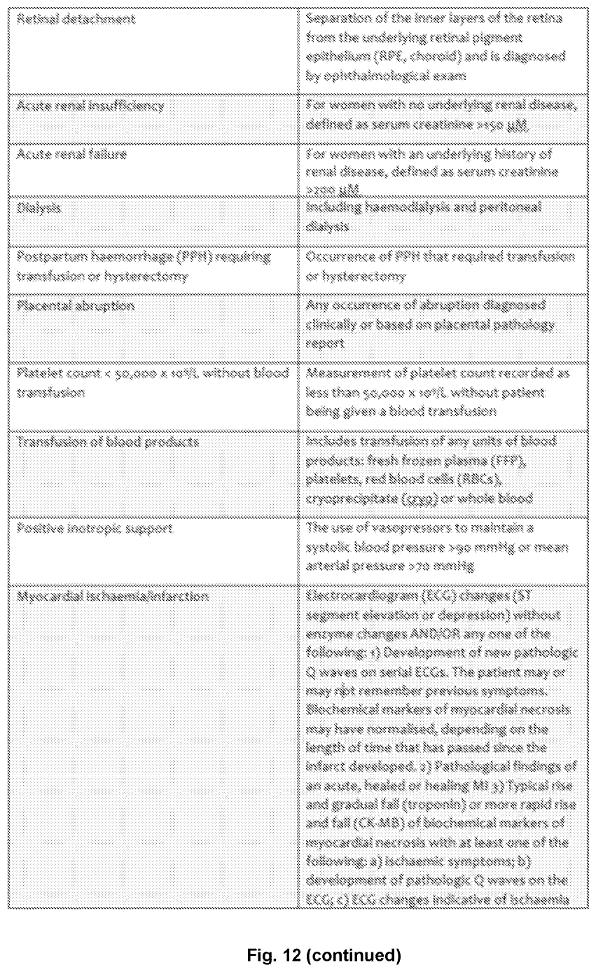

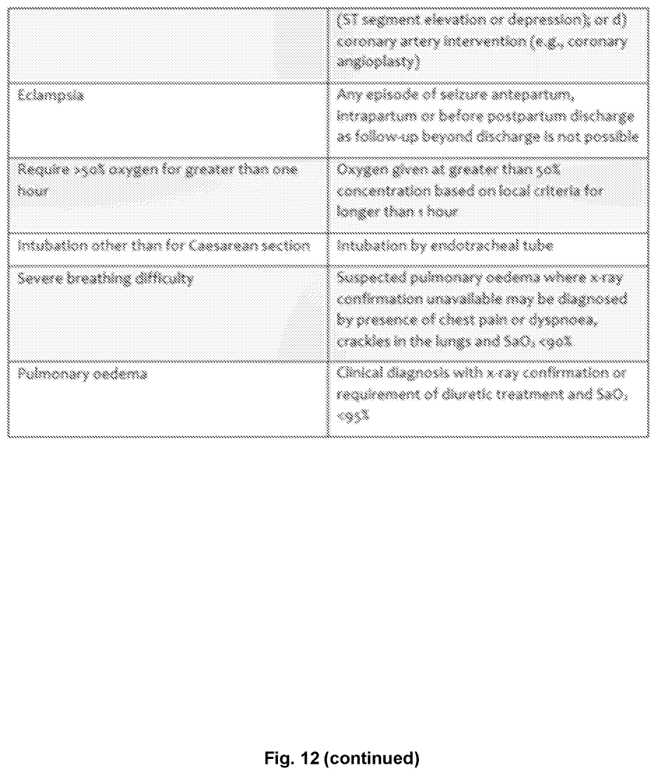

The primary study outcome was a composite developed by Delphi consensus and defined as the first occurrence of one or more of: maternal mortality or severe maternal morbidity (as listed in the table of FIG. 7 and defined in the table of FIG. 12), within two days of first assessment for pre-eclampsia.

### Missing Variables and Machine Learning

The most clinically-relevant abnormal value of each variable obtained during assessment on the first day of admission was taken to create a dataset, with only one observation per woman per day. Variables were excluded if at least 60% of values were missing.14,15 Variables with a lower proportion of missing values were included in multiple imputation if values were ‘missing at, or completely at, random’. As 2=19% of all data were missing, development and validation datasets were imputed 20 times each (number of imputations≥percentage of incomplete cases) using chained random forests (details of the missingness and the imputation process in supplementary statistical materials). Models were fitted on each development dataset; variables with above average importance using the Gini index were used. The model was tested on each imputed validation dataset; predictions were combined into a mean prediction per woman.

### Model Testing and Validation

The model's ability to classify women into outcome/no outcome groups, using the area-under-the-receiver-operator characteristic (AUROC) and calibration curves was assessed. Likelihood ratios were used to determine risk strata were data-defined, based on likelihood ratios, as follows: very-low risk (by a negative likelihood ratio <0.10), low risk (negative likelihood ratio of 0.1 to 0.2), high risk (positive likelihood ratio of 5.0 to 10.0), very-high risk (positive likelihood ratio greater than 10.0), and moderate risk otherwise.16 Positive likelihood ratios for very high and high risk were calculated by splitting the testing data into “very-high risk” and “not very high risk”, and “high risk” and “less than high risk” groups, then calculating likelihood ratios for a two group prediction using sensitivity and specificity (https://www.cebm.ox.ac.uk/resourcesiebm-tools/likelihood-ratios?11491492-1014-11ed-8177-0ab1d90019e6). Similarly, negative likelihood ratios for very low and low risk were calculated by creating “very-low risk” and “not very low risk”, and “low risk” and “higher than low risk” groups.

### Sensitivity Analyses

In sensitivity analyses, additional coagulation-related variables were included (as they are costly in all health systems), and mean platelet volume was excluded (as it is not routinely reported by all haematology laboratories). Secondary analyses were undertaken to predict outcomes (i) at seven days and any time following admission until primary hospital discharge, and (ii) limited to eclampsia or stillbirth. As some renal and haematological measures were included in both the candidate variables and the definitions of components of the combined maternal outcomes, we assessed the performance of the model in women who experienced no renal, no haematological, or neither renal nor haematological outcomes. The influence of multiple imputation on predictions was assessed by complete case analysis.

Statistics All analyses were performed using RStudio; multiple imputation was carried out using the missRanger package. Random forests were fitted on each dataset using the caret package with 10-fold cross-validation using the “rf” method, and combined into one ensemble model using the caretEnsemble package. Chi-squared and Fisher's exact, and

Mann-Whitney U tests were used for categorical and continuous variables, respectively, with statistical significance set at p<0.05.

### Further Results

Data were available for 8843 eligible women with pre-eclampsia, recruited from 53 institutions in 11 countries, with a median of 1244 [interquartile range, 693.5 to 1983.5] women per included cohort.

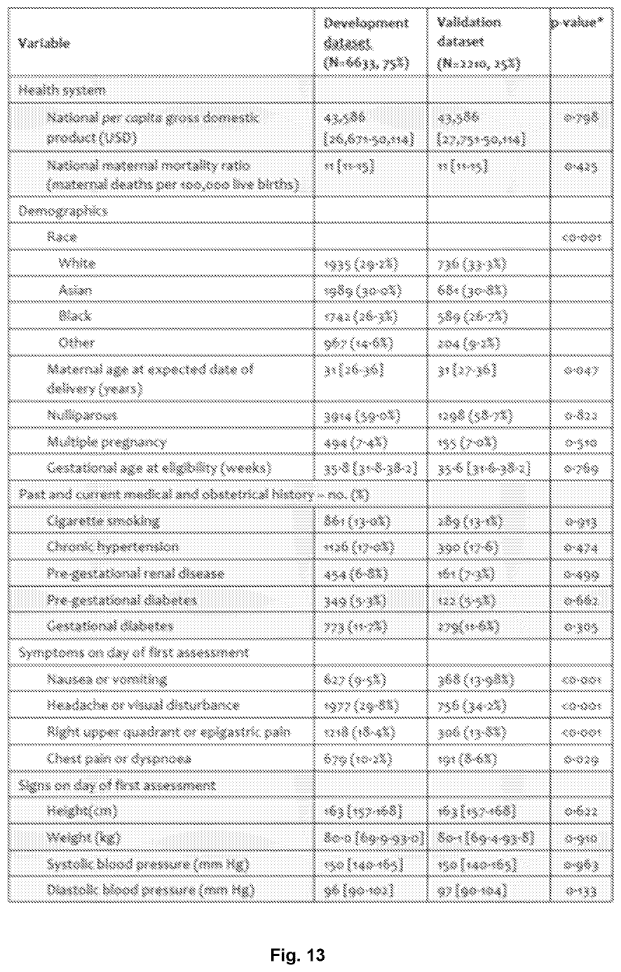

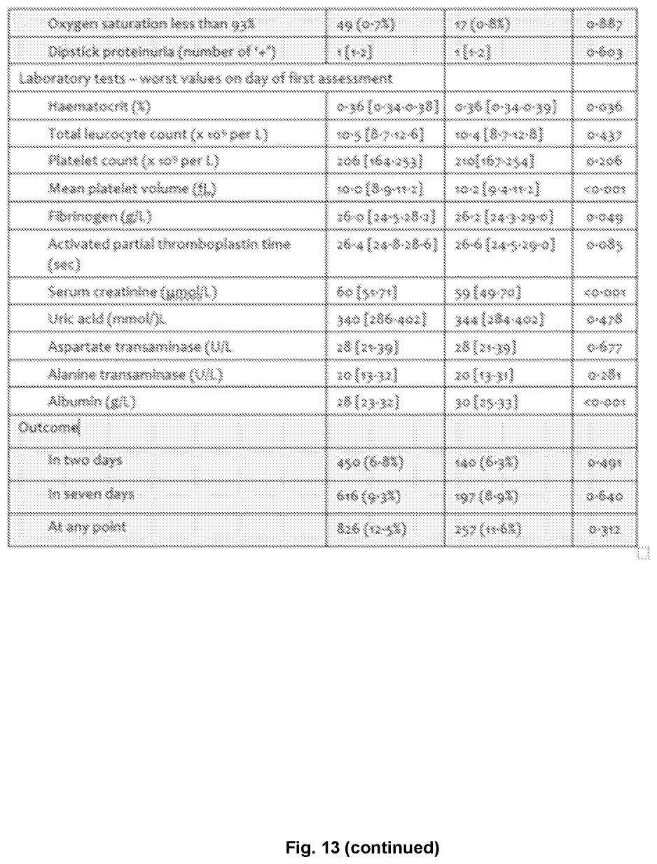

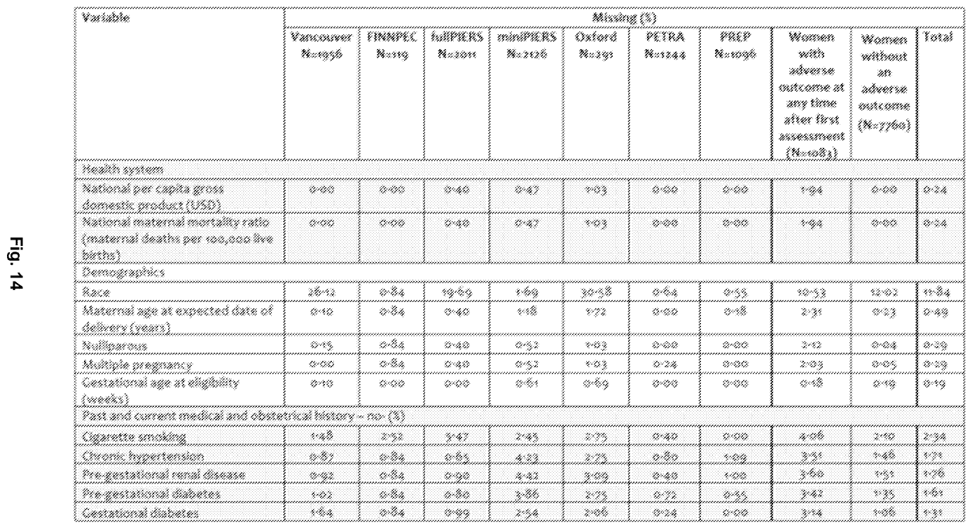

The table of FIG. 6, shows that the health system and individual-level characteristics of women who experienced an adverse maternal outcome differed from women who did not (development and validation cohort characteristics are presented in the table of FIG. 13). Women who experienced adverse outcomes were more often cared for in countries with lower per capita gross domestic products. These women were younger, more likely to have a multiple pregnancy, presented at an earlier gestational age; their past history was less often complicated by chronic hypertension, and their pregnancies by gestational diabetes. The study population had similar proportions of women from White, Asian, or Black ethnic backgrounds, regardless of complications.

At presentation with pre-eclampsia, women who subsequently developed an adverse maternal outcome differed in their symptom profile, signs, and laboratory results, although results largely overlapped (Table of FIG. 6). These women were more often symptomatic, had lower weight, higher blood pressure, lower oxygen saturation, higher dipstick proteinuria, and more perturbed laboratory results; differences in the latter were not changed significantly by imputation. Women who subsequently developed an adverse outcome more often received antenatal corticosteroids, antihypertensives, and magnesium sulphate. Babies of women who experienced adverse outcomes were born earlier and of lower birthweight, and more often died.

The table of FIG. 7 shows that 590 women (6.7%) had an adverse maternal outcome within two days of first assessment, 813 (9.2%) within seven days, and 1083 (12.2%) at any time prior to primary discharge. Most adverse outcomes were cardiorespiratory, haematological, hepatic, or placental. There were two maternal deaths, neither within 48 hours of first assessment.

### Model Development

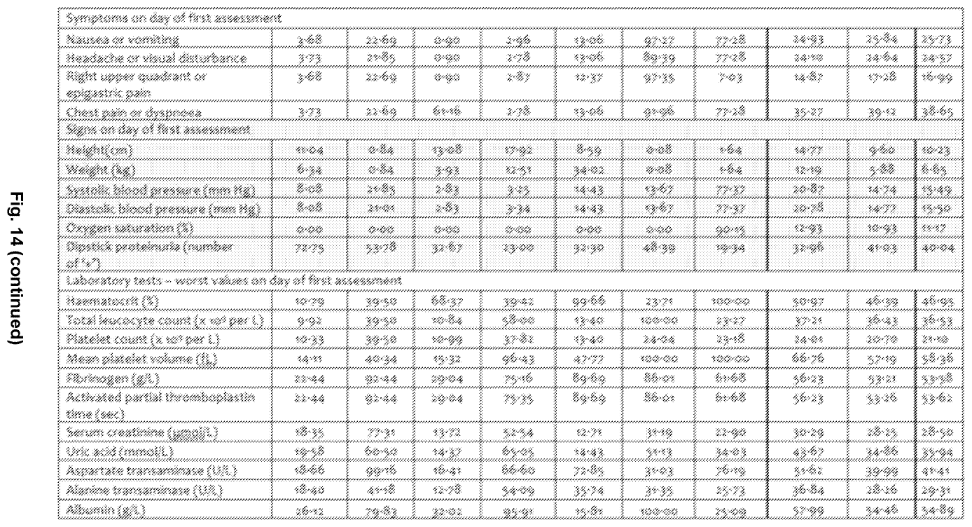

Data from a random 6633 women were used for model development. Total bilirubin, urinary protein to creatinine ratio, international normalised ratio, and lactate dehydrogenase were excluded given greater than 60% missingness (the Table of FIG. 14). The remaining variables considered either contained no missing or fewer than 60% ‘missing at random’ or ‘missing completely at random’ observations.

FIG. 5 shows the relative importance of the 18 PIERS-AI model variables. Platelet count and oxygen saturation were of greatest and least importance, respectively. All target organ systems were included, with following numbers of covariates: cardiorespiratory (N=3), renal (N=3), hepatic (N=2), and haematological (N=4). In addition, there were variables representing health systems (N=2), demographic characteristics (N=2), and anthropometry (N=2).

### Model Validation

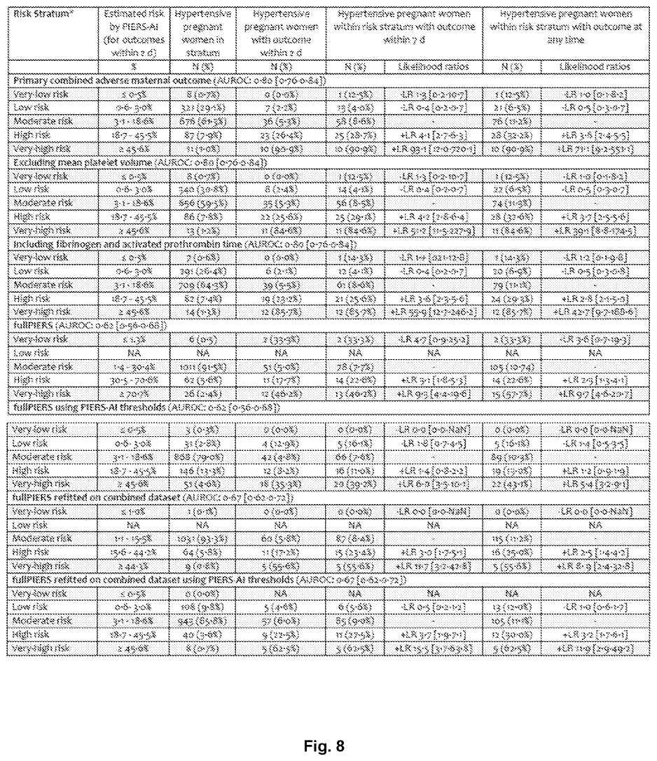

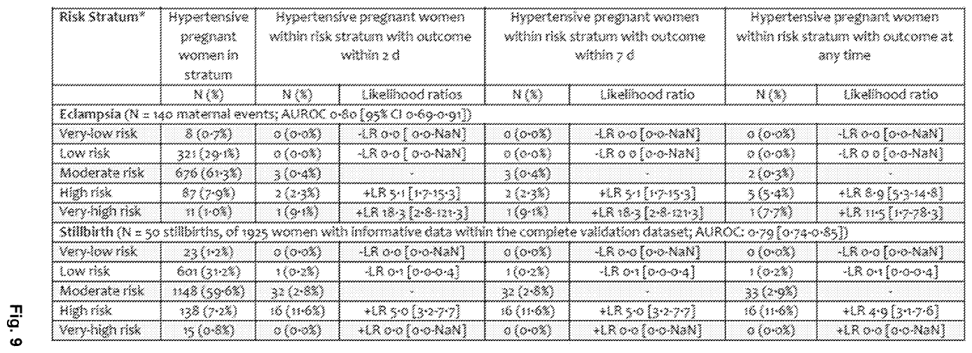

From the remaining 2210 women, 1105 informed selection of cut-off points for the risk groups according to likelihood ratios. The remaining 1105 women informed PIERS-AI model validation according to the selected cut-off points. PIERS-AI accurately stratified risk for adverse maternal outcomes within two days (Table of FIG. 8), with an AUROC of 0.80 [95% CI 0.76 to 0.84] (FIG. 5).

There were 329 (29.8%) women classified as being at very-low (8, 0.7%) or low risk (321, 29.1%) over the next two days (Table of FIG. 8). Among women at very-low risk, adverse maternal outcomes were very infrequent: 0 within two days, 1 (12.5%) each within seven days or at any time. Among women at low risk, adverse maternal outcomes were also infrequent: 7 (2.2%) within two days, 13 (4.0%) within seven days, and 21 (6.5%) at any time. PIERS-AI was insufficiently reassuring (negative likelihood ratio ≥0.2 for both very-low and low risk) within two-to-seven days or at any time.

There were 98 (8.9%) women classified as being at high (87, 7.9%) or very-high risk (11, 1.0%) of an adverse maternal outcome within two days (Table of FIG. 8). Among women at high risk, adverse maternal outcomes were frequent: 23 (26.4%) within two days, 25 (28.7%) within seven days, and 28 (32.2%) at any time. PIERS-AI could not sufficiently rule-in risks within 7 days or at any time (positive likelihood ratio≤5.0).

Among women at very-high risk, adverse maternal outcomes were very frequent, with all 10 (90.9%) occurring within two days. PIERS-AI was excellent at ruling-in risks (positive likelihood ratio >10.0) at any time.

Among the 676 (61.3%) women classified as being at moderate risk based on uninformative negative and positive likelihood ratios, adverse maternal outcomes occurred in 36 (5.3%) within two days, 58 (8.6%) within seven days, and 76 (11.2%) at any time (Table of FIG. 8).

### Sensitivity Analysis

Excluding mean platelet volume from the set of variables significantly altered the PIERS-AI model's performance, resulting in an AUROC of 0.80 [0.76, 0.84], 59.5% of women in the moderate risk stratum, and loss of reassurance for the very-low risk stratum (Table 3). Including fibrinogen and activated prothrombin time as variables neither improved the model's performance (AUROC: 0.80 [0.76, 0.84]) nor its risk stratification capacity (moderate risk stratum: 64.3%; Table of FIG. 8). The original fullPIERS model had poor performance in the combined validation dataset (Table of FIG. 8).

PIERS-AI had strong performance in ruling-out (very-low and low risk strata) and ruling-in (high, and very-high risk strata) subsequent seizures of eclampsia (Table of FIG. 9). For the 2210 women in the full validation dataset, stillbirth risks varied by likelihood ratio-based strata (Table of FIG. 9), such that women at very-low and low risk of adverse maternal events suffered few stillbirths; while those at moderate, high, and very-high risk suffered 2.8%, 11.6%, and 0.0% stillbirths, respectively (none reaching a positive likelihood ratio ≥5.0).

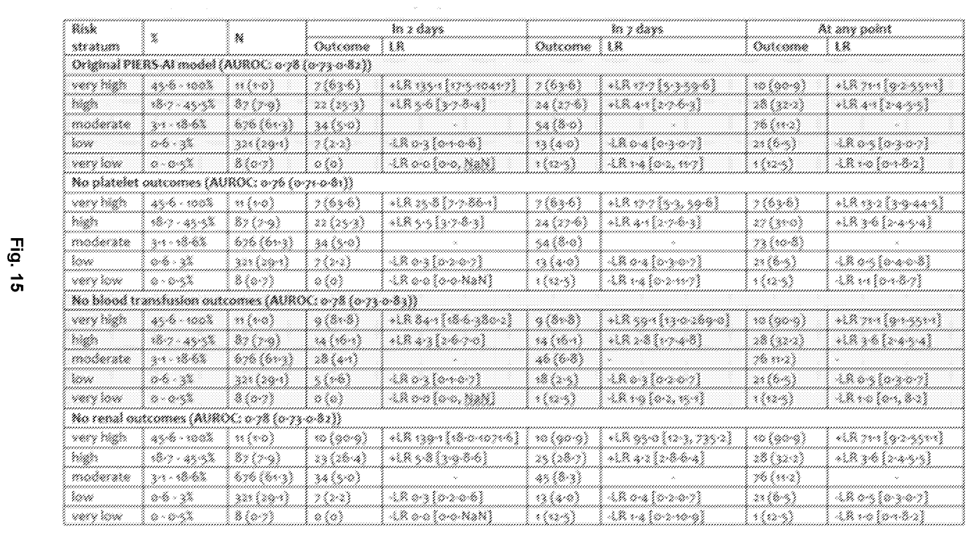

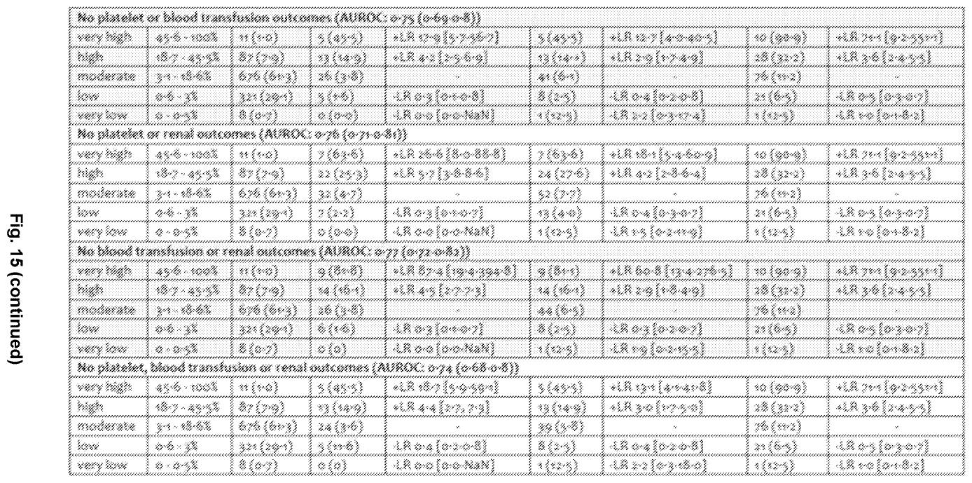

Removing renal and haematological components of the outcome did not significantly alter PIERS-AI performance (Table of FIG. 15). The complete case analysis showed a reduced AUROC, but with wider confidence intervals that included the AUROC of the imputed model (Table of FIG. 15).

### Further Comments

This study included almost 9000 women from 11 countries presenting for first assessment of pre-eclampsia, and used the random forest method to develop and validate an 18-variable model for maternal risk stratification, applicable for LMICs and HICs. The PIERS-AI model identifies nearly 40% of women with pre-eclampsia for whom care should be altered. The 29.8% of women (and their families and maternity care-providers) identified as being at very-low (0.7%) or low risk (29.1%) can be reassured that it is very unlikely that adverse maternal events will occur within two days. However, for the 8.9% of women identified to be a high (7.9%) or very-high risk (1.0%), a timely clinical response can be justified, based on a substantial risk of an adverse maternal event within two days, or for women at very-high risk, at any time. Identifying these women can inform discussions about place of care, transfer of care, antenatal and postnatal surveillance, co-interventions, and timed birth.

Strengths of our study include the large sample size-much larger than any previous study modelling adverse outcomes in women with pre-eclampsia. A list of variables with clinical external validity and availability, including all target organ systems, with the exception of the central nervous system were studied. Clinical central nervous system predictors rely on either the subjectivity of symptoms or the questionable reproducibility of deep tendon reflexes/clonus, particularly in pregnancy. Also, the PIERS-AI model does not include direct measures of coagulation; these are not routinely performed, and their addition neither altered model performance nor warranted related costs in women with pre-eclampsia. Artificial intelligence is suitable for managing a large number of variables, without assumption with respect to interactions and mediation, and addresses concerns regarding collinearity. Trade-offs were considered between model performance, complexity, and face validity.

The composite adverse maternal outcome for PIERS was Delphi-derived, similarly to the core maternal outcome list (iHOPE) for pre-eclampsia. However, there are differences: only PIERS includes uncontrolled hypertension, inotropic support, myocardial ischaemia or infarction, hepatic dysfunction, or transfusion, and only the iHOPE outcome set contains elevated liver enzymes, postpartum haemorrhage, and admission to intensive care. To confirm model performance, as some factors are both predictors and components of the combined outcome (e.g., serum creatinine, platelet count), we assessed PIERS-AI excluding renal or haematological components of the outcome, or both (Table of FIG. 15).

Multivariable model-based risk stratification of women with pre-eclampsia is recommended by national and international clinical practice guidelines. With access to full laboratory facilities, models have been based on either logistic regression or a survival model for time-to-adverse event. Women with a hypertensive disorder of pregnancy (including pre-eclampsia) in LMICs, without ready access to laboratory tests, benefit from the demographics-, symptom-, and signed-based miniPIERS model, with model performance improved by pulse oximetry.

The PIERS-AI model may improve on prior models in a number of ways. First, PIERS-AI is the first of model for risk stratification in pre-eclampsia that has been developed using machine learning from women with pre-eclampsia living in LMICs (sub-Saharan Africa, South America, South Asia, and Oceania-areas of the world where more than 99% of pre-eclampsia-related maternal mortality occurs) and HICs; we are unaware of another model that has included data from 11 less- and more-developed countries. Uniquely, the model includes national per capita gross domestic product and maternal mortality ratio; variables that adjust for location, avoiding adaptation of models to local outcome rates, particularly where information governance and research resources are absent or limited. Adjusting for local settings was required for original fullPIERS model validation in LMICs. The approach to create auto-adjustment for setting should be validated in further geographies. Second, PIERS-AI does not include maternal symptoms, criticised as a weakness of prior models, given the subjective nature, variable definitions, and inconsistent documentation of symptoms in health records. However, mean platelet volume, a marker of platelet consumption and release of immature platelet forms, is important within the PIERS-AI model. Haematology analysers routinely measure, but many laboratories do not report, mean platelet volume; the findings suggest mean platelet volume should be reported for all hypertensive pregnant women.

Third, although PIERS-AI does not include symptoms of central nervous system involvement, the model has clinically-relevant performance in identifying women at both least (very-low and low risk strata) and greatest (high and very-high risk strata) risk of developing eclampsia, especially within 7 days of initial assessment (Table of FIG. 9). These results could guide the targeted use of magnesium sulphate, and reduce the number-needed-to-treat, for the prevention of eclampsia. Depending on health system resilience, women in the moderate risk group may or may not be considered for magnesium sulphate prophylaxis. For women with disease onset before 34+0 weeks gestation, a loading dose of magnesium sulphate should be administered to reduce the risk of prematurity-related cerebral palsy.

Fourth, women in the very-low and low risk strata were very unlikely to suffer a stillbirth (Table of FIG. 9), and it is believed that such women can be appropriately reassured. All intrauterine fetal deaths were noted within two days of admission with pre-eclampsia. However, it was notable that the women in the high risk, and not the very-high risk, stratum bore the greatest risk of stillbirth, presumably as maternity care providers were sufficiently concerned by the condition of women in the very-high risk stratum to intervene for either maternal or fetal indications, or both, or in response to the woman experiencing an adverse maternal event.

### Data Missingness

The table of FIG. 14 shows the breakdown of missing data between women with and without adverse outcomes. Women who had an outcome had higher rates of missingness of GDP per capita, MMR, maternal age, parity, multiple pregnancy, all variables for past and current medical and obstetrical history, all variables for signs on day of admission bar dipstick proteinuria, haematocrit, platelet count, mean platelet volume, uric acid, AST, ALT, and albumin. They also had lower rates of missingness of right upper quadrant or epigastric pain, chest pain or dyspnoea, and dipstick proteinuria. Data were not missing equally between studies (Table of FIG. 14). Race was more likely to be missing in the Vancouver cohort, the fullPIERS cohort, and the Oxford cohort. Symptoms were rarely recorded within a day of first admission in the PETRA dataset and, except for right upper quadrant pain, in the PREP dataset. Blood pressure and oxygen saturation were missing for >70% on day of admission in the PREP data, however blood pressure was often recorded after day of admission, with 90% of women in the PREP data having blood pressure recorded on or the day after of admission. The highest rate of missingness was reported for the laboratory tests, some tests missing entirely from some datasets. Fibrinogen, activated partial thromboplastin time, aspartate transaminase and albumin were rarely measured in the FINNPEC data. Mean platelet volume and albumin were rarely measured in the miniPIERS data. In the Oxford data, haematocrit was not measured; fibrinogen and activated partial thromboplastin time were rarely measured. Total leucocyte count, mean platelet volume, and albumin were not measured, and fibrinogen and activated partial thromboplastin time were rarely measured in the PETRA data. Haematocrit and mean platelet volume were not measured in the PREP data. The fullPIERS data had the least amount of missing data. While rates of missingness were different between datasets and not all datasets recorded all variables, we assumed that patients within all cohorts were similar enough in their presentation that their observed and missing values for each variable would not be significantly different, or any potential difference can be accounted for by the other observed variables (such as gestational age). Datasets were combined on this assumption to create the data for model development and internal validation.

### Missing Data Imputation

While some machine learning methods have built in missing data imputation methods, many do not; hence it is important to handle missing data before applying machine learning methods which also ensures consistent data is used by each method. To do this, we first had to determine the mechanism of missingness for each variable with missing data. The mechanism of missingness can be missing completely at random (MCAR) when the missingness is completely by chance, missing at random (MAR) when missingness is related to some observed data but there is no relation between the observed and the unobserved data, or missing not at random (MNAR), where the unobserved data determines the missingness. Missing not at random was ruled out for all variables based on trial protocols and clinical input. Total bilirubin, urinary protein to creatinine ratio, international normalised ratio, and lactate dehydrogenase had >60% missingness and were excluded from consideration in our model.

To test if data in any given variable were missing at random, a corresponding missingness variable coded 0 if there is a value observed in the given variable and 1 if missing. A t-test or a chi-square test was performed (dependent on data type) on the missingness variable with all other predictor variables. If any of these tests is significant, the mechanism of missingness for the given variable would be missing at random. All variables had at least one significant t- or chi-square test, meaning that all variables were missing at random. Multiple imputation is generally suggested to be used for larger amounts of missing data; however, whether there is an amount of missingness that is too much for imputation in general is debatable. Previously it has been suggested that “too large” (such as 40% or 60%) amount of missing data should not be imputed, however recent studies suggest that variables with large amount of missingness can be imputed with minimal bias, given a large enough number of well defined imputations. It was decided not to impute variables missing for at least 60% of patients within a day of first admission. As we had a mixture of numeric and categorical predictor variables, multiple chained random forests was selected as the method of imputation. The missRanger R package for imputation, imputing all predictor variables with missing data under 60%, using all available predictor variables as auxiliary variables.

A rule of thumb for determining the number of imputations is to use at least the percentage of incomplete cases or more. In this case, as ~19% of all data were missing, development and validation datasets were imputed 20 times. To assess the impact of imputation on our model performance, a complete cases analysis was conducted.

Random forest is a supervised machine learning ensemble method that grows several classification/regression trees in order to compensate for overfitting and bias and derive a more accurate prediction model. The caret R package with 10-fold cross-validation was using the “rf” method was used to fit a random forest model on each imputed dataset. These random forest models were then combined into one ensemble model using the caretEnsemble R package.

Due to the nature of the growing process of a random forest, while it uses all available variables, it does not overfit as variables that do not significantly improve the predictive performance are given an importance of 0 and not used. Variables are assigned an importance based on their mean decrease in the Gini index. Variables with a high mean decrease in Gini index have a large effect on the outcome while variables with a mean decrease in Gini index close to zero have little to no effect on the outcome.

This means that while all available variables are used to grow the random forest, some variables may be redundant and thus could be removed without significantly affecting model performance. As random forests provide us with a way to assess a variable's importance in a model, we can use variable selection methods and model performance assessment to remove predictor variables and identify the model with the lowest number of variables that still provides accurate predictions.

The variables for our final model were selected by first growing a random forest model using all variables, ranking them by importance and choosing only variables with above average importance. This is simply done by calculating the mean of the MeanDecreaseGini (the mean decrease in the Gini index) of all variables and selecting variables for the new forest only if their MeanDecreaseGini is greater than or equal to this mean.

### Model Calibration

The calibration was assessed using multiple methods. The first method used was calibration-in-the-large, comparing the mean predicted probability (0.074) to the prevalence of the outcome in the dataset (0.067). Perfect calibration-in-the-large is an absolute difference of 0, the larger the absolute difference, the worse the calibration is. The direction of difference is also informative-a mean predicted probability greater than the prevalence suggests that the model overestimates the risk, while a mean predicted probability smaller than the prevalence suggests that the model underestimates the risk 10. As the absolute difference was 0.007 and mean predicted probability larger than the prevalence, we can see that the model slightly overestimates risk.

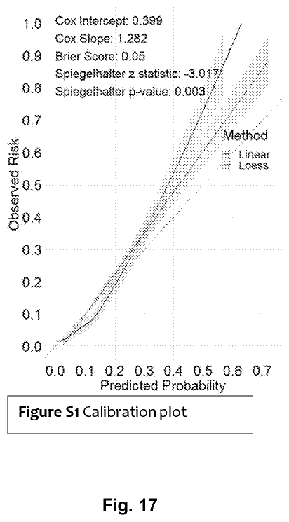

The second method used was the Cox calibration intercept and slope. Perfect calibration is a Cox calibration intercept of 0 and slope of 1. As seen in FIG. 17, the Cox Intercept for the model was 0.399, and the slope was 1.282. The Cox calibration intercept is also a measure of calibration-in-the-large, representing an overall bias in the predicted risk. The intercept of 0.399 shows an overestimation of risk, which is in line with the previous method of measuring calibration-in-the-large. The Cox calibration slope is a measure of spread, representing variability in the predicted probabilities. It should be noted that while the Cox calibration intercept can be interpreted on its own as a measure of calibration-in-the-large, the slope on its own does not measure calibration. A slope of 1 can be observed along with both good and poor calibration-in-the-large. As the slope measures the spread of the predicted probabilities, a slope less than 1 can be interpreted as the predicted probabilities varying too much compared to the observed outcomes, and a slope >1 as the predicted probabilities not varying as much as the observed outcomes. The slope of 1.261 shows that the model predicts a smaller range of probabilities than expected. The highest predicted probability in the validation dataset was p=0.724 and the smallest p=0.002.

Cox calibration fits a straight line through the predicted probability and observed proportion of outcomes pairs, which is useful for overall calibration, but does not show us in what ranges of predicted probabilities our model performs worse. In addition, there is a chance of the model overestimating risk in one area and underestimating risk in another resulting in a good Cox calibration. It can be assessed if the model performs equally well or poorly across all predicted probabilities by obtaining a flexible calibration curve using loess (a nonparametric regression method) function, as seen in FIG. 17. The loess calibration curve shows that the model overestimates risk for the lower predicted probabilities, and underestimates risk for the higher predicted probabilities.

The final method used was the Spiegelhalter z test (see FIG. 17) derived from the Brier score (the mean squared difference between the predicted probability and the actual outcome). This method allows us to calculate a z statistic for calibration, which then can be tested to show if the model is statistically significantly improperly calibrated. As the Spiegelhalter p-value was <0.05, the model was not well calibrated. Despite calibration being sub-optimal, we had chosen a priori to prioritise risk classification groups to give a more accurate prediction of risk. As the risk categories were determined based on likelihood ratios from the observed risk in the testing dataset, the selected threshold values account for the overestimation of small risk by moving the thresholds for very-low, low and moderate risk close together, and the underestimation of large risk by having wider ranges for the high and very-high risk groups.

### Complete Case Analysis

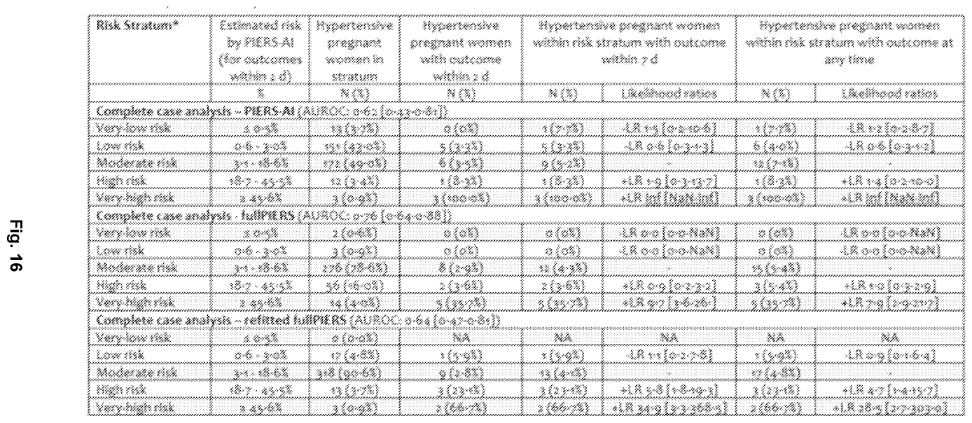

The table of FIG. 16 represents results for a complete case analysis of the PIERS-AI and fullPIERS models. Of the 2210 women in the validation dataset, 351 had no missing values for any of the PIERS-AI or fullPIERS variables (Table of FIG. 14). To test model performance with no imputations, the PIERS-AI model, the original fullPIERS model and the refitted fullPIERS model were all tested on these complete cases. The complete cases had a lower outcome rate (4.3% in 2 days, 5.4% in 7 days, 6.6% at any point) than the whole of the dataset (6.65, 9.1% and 12.2% respectively). This is in line with the previous observation that patients with an outcome were more likely to have missing values. The PIERS-AI model performed better on the complete observations than the imputed validation datasets.

1. A method of obtaining at least one of a risk level or a probability associated with one or more adverse maternal outcomes for a subject with suspected or confirmed preeclampsia comprising:
obtaining input data comprising at least subject data for the subject, wherein the subject data comprises a combination of at least two of: demographic data; vital sign data representative of one or more vital signs of the subject; and sample data representative of one or more parameters obtainable from an analysis of a sample;
providing the obtained input data to a machine learning derived procedure configured to obtain at least one of the risk level or probability associated with one or more adverse maternal outcomes for the subject, wherein the input data further comprises health care system data representing a value for at least one statistic or indicator associated with at least one of the health care system, a country of the health care system or a region of the health care system.

2. The method of claim 1, wherein obtaining input data comprises at least one of a), b), c), or d):
a) receiving user input data representative of at least one of the country or region of the health care system and retrieving the health care system data representing a value for at least one statistic associated with at least one of the country or region of the health care system;
b) performing a vital sign measurement to obtain the vital sign data;
c) receiving user input data representative of the demographic data;
d) obtaining a sample from the subject and performing a sample analysis on the sample to obtain the sample data, or, wherein the sample comprises a blood sample and wherein the sample analysis comprises a blood sample analysis to obtain at least one of: a) one or more haematological parameters including haematocrit, platelet count, total leukocyte count; or b) one or more renal parameters including lactate dehydrogenase, aspartate transaminase, alanine transaminase, serum albumin.

3. The method of claim 1, further comprising at least one of receiving user input data representing at least some input data or displaying at least one of the obtained risk level or probability.

4. The method of claim 1, wherein at least one of a), b), or c):
a) the obtained input data represent values for a reduced set of input variables, wherein the selected set of input variables are selected from a larger set of input variables in accordance with a feature reduction process;
b) wherein the input data further comprises input representing an elapsed time and wherein at least one of the risk level or probability is obtained in dependence on at least the elapsed time; or
c) performing one or more feature reduction processes comprising a recursive feature elimination process based on importance scores for a larger set of parameters.

5. The method of claim 1, wherein the health care system data comprises at least one non-clinical statistic or indicator.

6. The method of claim 1, wherein the health system data comprises at least one of a national or regional per capita gross domestic product or a national or regional maternal mortality ratio.

7. The method of claim 1, wherein the input data are representative of values for a set of selected set of input features, wherein:
a) the health care system data are representative of: national per capita gross domestic product and national or regional maternal mortality ratio;
b) the demographic data are representative of: maternal age at expected date of delivery, gestational age at eligibility;
c) the vital sign data are representative of: height, weight at time of assessment, systolic blood pressure, diastolic blood pressure, oxygen saturation; and
d) the sample data are representative of: haematology sample data comprising haematocrit, platelet count, total leukocyte count; renal sample data comprises: serum creatinine, uric acid; hepatic sample data comprising: lactate dehydrogenase, aspartate transaminase, alanine transaminase, serum albumin.

8. The method of claim 1, wherein obtaining the input data comprises:
obtaining sample data for a first time and providing the sample data as part of first input data to the model to obtain at least one of the risk level or probability associated with the first time and obtaining sample data for a second, subsequent time and providing the sample data as part of the input data to the model to obtain at least one of the risk level or probability associated with the second time.

9. The method of claim 1, wherein the machine learning derived procedure comprises at least one part configured to take into account a magnitude and rate of change of the obtained input data.

10. The method of claim 1, wherein at least one of a) the machine learning derived procedure comprises a combination of one or more of random forest or regression models with a generalised linear mixed model; or b) the machine learning derived procedure comprises a combination of a Bayesian generalized linear mixed model (GLMM) and a random forest model.

11. The method of claim 1, wherein the input data does not include symptom data representative of one or more symptoms of the subject.

12. The method of claim 1, wherein the input data comprises data representing values for at least the following input variables: national per capita gross domestic product, maternal mortality ratio, systolic and diastolic blood pressure, uric acid.

13. The method of claim 1, wherein the one or more feature reduction processes comprises a recursive feature elimination process based on importance scores for the larger set of parameters.

14. (canceled)

15. The method of claim 1, wherein at least one of a), b), or c):
a) at least one of the risk level or probability represents at least one of the risk level or probability of an occurrence of the adverse maternal outcome within one or more predefined time periods, or, wherein the predefined time period comprises at least one of two days or seven days;
b) the adverse maternal outcome comprises at least one of: maternal death; an adverse central nervous system event; a cardiorespiratory event; a hematologic event; a hepatic event; a renal event or one or more of: placental abruption, severe ascites, bell's palsy; or
c) the machine learning derived procedure is configured to classify the subject into one of a plurality of risk levels based on a probability of the occurrence of one or more adverse maternal events in a predetermined time period.

16.-17. (canceled)

18. A method of training a machine learning derived procedure comprising:
obtaining training data associated with a plurality of subjects, wherein the training data comprises subject data, the subject data comprising a combination of at least two of: health system data; demographic data; symptom data representative of the presence of one or more symptoms; vital sign data representative of one or more vital signs of the subject and sample data representative of one or more parameters obtainable using an analysis of a sample; and
performing a training process using at least some of the training data to obtain a trained machine learning derived procedure configured to receive further input data comprising at least further subject data for a further subject with suspected or confirmed preeclampsia and obtain at least one of a risk level or a probability associated with one or more adverse maternal outcomes for the subject,
wherein the training data and further input data comprises health care system data representing a value for at least one statistic associated with at least one of the health care system, a country of the health care system or a region of the health care system.

19. The method of claim 14, wherein at least one of a) or b):
a) the training process comprises performing a feature reduction process to select a reduced set of input variables from a larger set of input variables and training the machine learning derived procedure to receive input data representative of values for the reduced set of input variables; or
b) wherein the training data is representative of data obtained over a time period, such that the trained machine learning derive procedure is configured to receive input representative of an elapsed time and obtain at least one of the risk level or probability is obtained in dependence on at least the elapsed time.

20. The method of claim 19, wherein the feature reduction process comprises:
training one or more models using first data representative of a first group of features;
determining importance scores for each feature of the first group of features; and
selecting the reduced set of features based on the determined importance scores.

21. The method of claim 18, wherein the training method comprises performing an iterative process comprising:
a) fitting a plurality of models of a first type using training data for a plurality of subjects over a plurality of time points using a target outcome;
b) fitting at least one model of a second type using at least predictions obtained from the plurality of models of the first type models and the target outcome; and
c) using an output from the second model to update the target outcome for fitting the plurality of models of the first type, or wherein the first type comprises a random forest based model and the second type comprises a generalized linear mixed model.

22. The method of claim 18, wherein at least one of a) or b):
a) the training data comprises at least one of: training data for subjects with pre-eclampsia prior to 34 weeks gestation; or training data for subjects in sub-Saharan Africa, North and South America, South Asia, Europe and Oceania; or
b) the method further comprises identifying missing data and replacing missing data using a missing data replacement scheme.

23. (canceled)

24. A system comprising:
one or more processors; and
one or more tangible, non-transitory memories configured to communicate with the one or more processors,
the one or more tangible, non-transitory memories having instructions stored thereon that, in response to execution by the one or more processors, cause the one or more processors to perform operations comprising:
obtain input data comprising at least subject data for the subject, wherein the subject data comprises a combination of at least two of: demographic data; vital sign data representative of one or more vital signs of the subject; and sample data representative of one or more parameters obtainable from an analysis of a sample;
provide the obtained input data to a machine learning derived procedure configured to obtain at least one of the risk level or probability associated with one or more adverse maternal outcomes for the subject, wherein the input data further comprises health care system data representing a value for at least one statistic or indicator associated with at least one of the health care system, a country of the health care system or a region of the health care system.

25. (canceled)
